(19) United States
(12) Patent Application Publication
(10) Pub. No.: US 2005/0277627 A1
(43) Pub. Date: Dec. 15, 2005

(54) Colchinol derivatives as vascular damaging agents

(76) Inventors: Jean Claude Arnould, Reims (FR); Maryannick Andree Lamorlette, Reims (FR)

Correspondence Address: MORGAN LEWIS & BOCKIUS LLP, 1111 PENNSYLVANIA AVENUE NW, WASHINGTON, DC, 20004, US

(21) Appl. No.: 10/332,129

(22) PCT Filed: Jul. 4, 2001
(86) PCT No.: PCT/GB01/02966

§ 371 Date: Jan. 6, 2003

(30) Foreign Application Priority Data
Jul. 7, 2000 (EP) 00401978.2

(51) Int. Cl. A61K 31/55

(52) U.S. Cl. 514/217

(57) ABSTRACT

The invention relates to colchinol derivatives of the formula 1: wherein the substituents are as defined in the description or a pharmaceutically-acceptable salt, solvate or pro-drug thereof. The invention also relates to processes for preparing compounds of formula (1), pharmaceutical compositions of compounds of formula (1) and the use of compounds of formula (1), in the manufacture of a medicament for use in the production of a vascular damaging effect in a warm blooded animal.

The present invention relates to vascular damaging agents, to the use of compounds of the invention in the manufacture of medicaments for use in the production of antiangiogenic effects in warm-blooded animals such as humans, to processes for the preparation of such compounds, to pharmaceutical compositions containing such compounds as active ingredient, to methods for the treatment of disease states associated with angiogenesis and to the use of such compounds as medicaments.

Normal angiogenesis plays an important role in a variety of processes including embryonic development, wound healing and several components of female reproductive function. Undesirable or pathological angiogenesis has been associated with disease states including diabetic retinopathy, psoriasis, cancer, rheumatoid arthritis, atheroma, Kaposi's sarcoma and haemangioma (Fan et al, 1995, Trends Pharmacol. Sci. 16: 57-66; Folkman, 1995, Nature Medicine 1: 27-31). Formation of new vasculature by angiogenesis is a key pathological feature of several diseases (J. Folkman, New England Journal of Medicine 333, 1757-1763 (1995)). For example, for a solid tumour to grow it must develop its own blood supply upon which it depends critically for the provision of oxygen and nutrients; if this blood supply is mechanically shut off the tumour undergoes necrotic death. Neovascularisation is also a clinical feature of skin lesions in psoriasis, of the invasive pannus in the joints of rheumatoid arthritis patients and of atherosclerotic plaques. Retinal neovascularisation is pathological in macular degeneration and in diabetic retinopathy.

Reversal of neovascularisation by damaging the newly-formed vascular endothelium is expected to have a beneficial therapeutic effect. The present invention is based on the discovery of tricyclic compounds that surprisingly specifically damage newly formed vasculature without affecting the normal, established vascular endothelium of the host species, a property of value in the treatment of disease states associated with angiogenesis such as cancer, diabetes, psoriasis, rheumatoid arthritis, Kaposi's sarcoma, haemangioma, acute and chronic nephropathies, atheroma, arterial restenosis, autoimmune diseases, acute inflammation, endometriosis, dysfunctional uterine bleeding and ocular diseases with retinal vessel proliferation.

Compounds of the present invention are colchinol derivatives. Colchinol derivatives for example N-acetyl-colchinol are known. Anti-tumour effects have been noted on animal models (see for example—Jnl. Natl. Cancer Inst. 1952, 13, 379-392). However, the effect studied was that of gross damage (haemorrhage, softening and necrosis) and there is no suggestion of treatment of inappropriate angiogenesis by destruction of neovasculature.

It is believed, though this is not limiting on the invention, that the use of compounds of the invention damages newly-formed vasculature, for example the vasculature of tumours, thus effectively reversing the process of angiogenesis as compared to known anti-angiogenic agents which tend to be less effective once the vasculature has formed.

According to one aspect of the present invention there is provided a compound of the formula I: 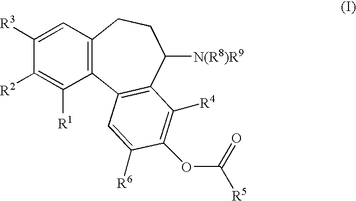
wherein:

R¹, R² and R³ are each independently hydroxy, phosphoryloxy (—OPO₃H₂), C₁₋₄alkoxy or an in vivo hydrolysable ester of hydroxy; with the proviso that at least two of R¹, R² and R³ are C₁₋₄alkoxy;

R⁴ and R⁶ are each independently selected from:

hydrogen, nitro, amino, N—C₁₋₄alkylamino, N,N-di(C₁₋₄alkyl)amino, hydroxy, fluoro, C₁₋₄alkoxy and C₁₋₄alkyl;

R⁵ is selected from one of the following groups:

1) of the formula -A-X¹—Y¹—B, wherein:

A is C₁₋₄alkylene or —(CH₂)ₚ-Q- (wherein p is 0, 1 or 2 and Q is phenylene or thienylene);

X¹ is —O—, —CO—, —C(O)O—, —CON(R¹⁰)—, —N(R¹⁰)—, —N(R¹⁰)CO—, N(R¹⁰)C(O)O—, —N(R¹⁰O)CON(R¹¹)—, —N(R¹⁰)SO₂—, —SO₂N(R¹⁰)— or OC(O)N(R¹⁰)—

(wherein R¹⁰ is hydrogen, C₁₋₃alkyl, hydroxyC₂₋₃alkyl, aminoC₂₋₃alkyl or C₁₋₃alkoxyC₂₋₃alkyl);

Y¹ is C₁₋₃alkylene;

B is carboxy, sulpho, phosphoryloxy, hydroxy, amino, N—(C₁₋₄alkyl)amino, N,N-di(C₁₋₃alkyl)amino (wherein the C₁₋alkyl group in the alkylated amino groups is optionally substituted by hydroxy or amino), —R¹² or —NHC(R¹³)COOH;

(wherein R¹² is a 5-6-membered saturated heterocyclic group (linked via carbon or nitrogen) containing 1 or 2 ring heteroatoms, selected independently from O, S and N, which heterocyclic group is optionally substituted by 1 or 2 substituents selected from:
oxo, hydroxy, halogeno, C₁₋₄alkyl, C₂₋₄alkanoyl, carbamoyl, N—C₁₋₄alkylcarbamoyl, N,N-di-(C₁₋₄alkyl)carbamoyl, hydroxyC₁₋₄alkyl, C₁₋₄alkoxy, cyanoC₁₋₃alkyl, carbamoylC₁₋₃alkyl, carboxyC₁₋₄alkyl, aminoC₁₋₄alkyl, N,N-di(C₁₋₄alkyl)aminoC₁₋₄alkyl, C₁₋₄alkoxyC₁₋₄alkyl, C₁₋₄alkylsulphonylC₁₋₄alkyl and R¹⁴ (wherein R¹⁴ is a 5-6-membered saturated heterocyclic group (linked via carbon or nitrogen) containing 1 or 2 ring heteroatoms, selected independently from O, S and N, which heterocyclic group is optionally substituted by 1 or 2 substituents selected from:
   oxo, hydroxy, halogeno, C₁₋₄alkyl, hydroxyC₁₋₄alkyl, C₁₋₄alkoxy, C₁₋₄alkoxyC₁₋₄alkyl and C₁₋₄alkylsulphonylC₁₋₄alkyl);

R¹³ is an amino acid side chain;

2) of the formula: 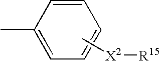
wherein: the phenyl ring is substituted by —X²—R¹⁵ in the 3- or 4-position;

X² is —CO— or of the formula —(CH₂)ᵣ— (wherein r is 0, 1, 2 or 3) and R¹⁵ is a 5-6 membered saturated heterocyclic group (linked via a ring carbon or nitrogen atom) containing 1 or 2 ring heteroatoms selected from O, S and N, which heterocyclic group is optionally substituted by 1 or 2 substituents selected from
oxo, hydroxy, halogeno, C₁₋₄alkyl, C₂₋₄alkanoyl, carbamoyl, N—C₁₋₄alkylcarbamoyl, N,N-di-(C₁₋₄alkyl)carbamoyl, hydroxyC₁₋₄alkyl, C₁₋₄alkoxy, cyanoC₁₋₃alkyl, carbamoylC₁₋₃alkyl, carboxyC₁₋₄alkyl, C₁₋₄aminoalkyl, N,N-di(C₁₋₄alkyl)aminoC₁₋₄alkyl, C₁₋₄alkoxyC₁₋₄alkyl, C₁₋₄alkylsulphonylC₁₋₄alkyl and R¹⁴ (wherein R¹⁴ is as hereinabove defined);

provided that the heterocyclic group (R¹⁵) is substituted by at least one substituent selected from C₂₋₄alkanoyl, carbamoyl, N—C₁₋₄alkylcarbamoyl and N,N-di(C₁₋₄alkyl)carbamoyl;

3) —(CH₂)ₐ—Y²—(CH₂)_{b}—R¹⁵ (wherein a is 0, 1, 2, 3 or 4; b is 0, 1, 2, 3 or 4; Y² is a direct bond, —O—, —C(O)—, —N(R¹⁶)—, —N(R¹⁶)C(O)— or —C(O)N(R¹⁶)— (wherein R¹⁶ is hydrogen, C₁₋₃alkyl, hydroxyC₂₋₃alkyl, aminoC₂₋₃alkyl or C₁₋₃alkoxyC₂₋₃alkyl) and wherein 1 or 2 of the (CH₂)ₐ or (CH₂)_{b} groups are optionally substituted by 1 or 2 substituents selected from hydroxy and amino and R¹⁵ is as hereinabove defined; provided that when a is 0, then y² is a single direct bond;

4) N,N-di(C₁₋₄alkyl)carbamoylC₁₋₄alkyl- (wherein the alkyl groups are independently optionally substituted by 1 or 2 substituents selected from:
amino, N—C₁₋₄alkylamino, N,N-di(C₁₋₄alkyl)amino, hydroxy, C₁₋₄alkoxy, C₁₋₄alkanoyl, carboxy, sulpho and phosphoryloxy);

provided that:

a) when A is C₁₋₄alkylene and X¹ is of the formula —CO—, —N(R¹⁰)—, —N(R¹⁰)CO— or —CON(R¹⁰)— then when B is R¹², R¹² is as defined hereinabove for R¹⁵;

b) when A is C₁₋₄alkylene and X¹ is of the formula —N(R¹⁰)CO—, —CON(R¹⁰)—, or —C(O)O—, then B is not carboxy;

c) when A is C₁₋₄alkylene and X¹ is —CONH— or —NHCO—, then B is not carboxy, hydroxy, phosphoryloxy, amino, N—C₁₋₄alkylamino or N,N-di-C₁₋₄alkylamino;

R^{S} is a group —Y³R¹⁷ (wherein Y³ is a direct bond, —C(O)—, —C(O)O—, —N(R¹⁸)—, —C(O)N(R¹⁸)—, —SO₂— or —SO₂NR¹⁸— wherein R¹⁸ is hydrogen, C₁₋₃alkyl, hydroxyC₂₋₃alkyl, aminoC₂₋₃alkyl or C₁₋₃alkoxyC₂₋₃alkyl) and R¹⁷ is selected from one of the following 4 groups:

1) hydrogen, C₁₋₄alkyl, phenyl, C₁₋₄alkylY⁴C₁₋₄alkyl (wherein Y⁴ is —C(O)—, —NR¹⁹C(O)— or —C(O)NR¹⁹— (wherein R¹⁹ is hydrogen, C₁₋₃alkyl, hydroxyC₂₋₃alkyl, aminoC₂₋₃alkyl or C₁₋₃alkoxyC₂₋₃alkyl));

[which alkyl, alkylY⁴alkyl or phenyl group is optionally substituted by 1 or 2 substituents selected from:

halogeno, amino, N—C₁₋₄alkylamino, N,N-di(C₁₋₄alkyl)amino, hydroxy, carboxy, —CON(R²³)R²⁴ (wherein R²³ and R²⁴ are independently selected from hydrogen, C₁₋₃alkyl, hydroxyC₂₋₃alkyl, aminoC₂₋₃alkyl and C₁₋₃alkoxyC₂₋₃alkyl), C₁₋₄alkoxy, C₁₋₄alkoxycarbonylamino, C₁₋₄alkanoyl, sulpho, phosphoryloxy, R¹² (wherein R¹² is as hereinabove defined), and a group —Y⁵R²⁰ [wherein Y⁵ is —NR²¹C(O)— or —OC(O)— (wherein R²¹ represents hydrogen, C₁₋₃alkyl or C₁₋₃alkoxyC₂₋₃alkyl) and R²⁰ is C₁₋₄alkyl or a group R²² (wherein R²² is a 5 or 6 membered aromatic heterocyclic group containing 1 to 4, inclusive, ring heteroatoms selected independently from O, N and S, which aromatic heterocyclic group is optionally substituted by 1 or 2 substituents selected from hydroxy, amino, C₁₋₄alkyl, aminoC₁₋₄alkyl, N—C₁₋₄alkylamino₁₋₄alkyl, N,N-di(C₁₋₄alkyl)aminoC₁₋₄alkyl, carboxy, —CONR²⁵R²⁶ and —NR²⁵COR²⁷ (wherein R²⁵ and R²⁶, which may be the same or different, are hydrogen, C₁₋₃alkyl, hydroxyC₂₋₃alkyl, aminoC₂₋₃alkyl or C₁₋₃alkoxyC₂₋₃alkyl and R²⁷ is C₁₋₃alkyl, hydroxyC₂₋₃alkyl, aminoC₂₋₃alkyl or C₁₋₃alkoxyC₂₋₃alkyl))];

2) R²² (wherein R²² is as hereinabove defined);

3) R²²—C₁₋₄alkyl- (wherein R²² is as hereinabove defined); or

4) R¹²Y⁷C₁₋₄alkyl- (wherein R¹² is as hereinabove defined and Y⁷ is —C(O)—, —NR²³C(O)—, —NR²³C(O)C₁₋₄alkyl-, —C(O)NR²³— or —C(O)NR²³C₁₋₄alkyl- (wherein R²³ is as hereinabove defined))];

and R⁹ is hydrogen or C₁₋₃alkyl;

or a pharmaceutically-acceptable salt, solvate or pro-drug thereof.

In another aspect, the invention relates to a compound of the formula (I) as hereinabove defined or to a pharmaceutically-acceptable salt thereof.

In this specification the generic term “alkyl” includes both straight-chain and branched-chain alkyl groups. However references to individual alkyl groups such as “propyl” are specific for the straight-chain version only and references to individual branched-chain alkyl groups such as “isopropyl” are specific for the branched-chain version only. An analogous convention applies to other generic terms.

R¹³ is an amino acid side chain. This includes amino acid side chains from natural and non-natural amino acids and includes the possibility of R¹³ joining to the NH group so as to form a ring as in the amino acid proline. It includes α-amino acids β-amino acids and γ-amino acids. In addition, the amino acids may be L-isomers or D-isomers, but preferably L-isomers. Preferred amino acids include glycine, alanine, valine, leucine, isoleucine, methionine, proline, phenylalanine, tryptophan, serine, threonine, cysteine, tyrosine, asparaginine, glutamine, aspartic acid, glutamic acid, lysine, arginine, histidine, β-alanine and ornithine. More preferred amino acids include glutamic acid, serine, threonine, arginine, glycine, alanine, β-alanine and lysine. Especially preferred amino acids include glutamic acid, serine, threonine, arginine, alanine and β-alanine. Specific values for R¹² include hydrogen, C₁₋₄alkyl, C₁₋₄alkylthioC₁₋₄alkyl, hydroxyC₁₋₄alkyl, thioC₁₋₄alkyl, phenylC₁₋₄alkyl (optionally substituted by hydroxy), guanidinoC₁₋₄alkyl, carboxyC₁₋₄alkyl, carbamoylC₁₋₄alkyl, aminoC₁₋₄alkyl and imidazolyl C₁₋₄alkyl and R¹² forming a pyrrolidinyl ring with the NH group. Preferred values for R¹³ include hydrogen, C₁₋₄alkyl, C₁₋₄alkylthioC₁₋₄alkyl, hydroxyC₁₋₄alkyl, thioC₁₋₄alkyl, guanidinoC₁₋₄alkyl, carboxyC₁₋₄alkyl, carbamoylC₁₋₄alkyl and aminoC₁₋₄alkyl.

In this specification, the term heteroaryl is used to describe fully saturated heterocyclic rings. Examples of 5- or 6-membered heteroaryl rings include pyrrolyl, imidazolyl, pyrazolyl, isothiazolyl, isoxazolyl, pyridyl, pyrazinyl, pyrimidinyl, pyridazinyl, triazolyl, tetrazolyl, thiazolyl, thiadiazolyl, thienyl, furyl and oxazolyl.

It is to be understood that, insofar as certain of the compounds of Formula I defined above may exist in optically active or racemic forms by virtue of one or more asymmetric carbon atoms, the invention includes in its definition any such optically active or racemic form which possesses vascular damaging activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

Suitable values for the generic radicals referred to above include those set out below. Within the present invention it is to be understood that a compound of the formula I or a salt thereof may exhibit the phenomenon of tautomerism and that the formulae drawings within this specification can represent only one of the possible tautomeric forms. It is to be understood that the invention encompasses any tautomeric form which has vascular damaging activity and is not to be limited merely to any one tautomeric form utilised within the formulae drawings.

It is also to be understood that certain compounds of the formula I and salts thereof can exist in solvated as well as unsolvated forms such as, for example, hydrated forms. It is to be understood that the invention encompasses all such solvated forms which have vascular damaging activity.

The present invention relates to the compounds of formula I as hereinbefore defined as well as to the salts thereof. Salts for use in pharmaceutical compositions will be pharmaceutically acceptable salts, but other salts may be useful in the production of the compounds of formula I and their pharmaceutically acceptable salts. Pharmaceutically acceptable salts of the invention may, for example, include acid addition salts of the compounds of formula I as hereinbefore defined which are sufficiently basic to form such salts. Such acid addition salts include for example salts with inorganic or organic acids affording pharmaceutically acceptable anions such as with hydrogen halides (especially hydrochloric or hydrobromic acid of which hydrochloric acid is particularly preferred) or with sulphuric or phosphoric acid, or with trifluoroacetic, citric or maleic acid. Suitable salts include hydrochlorides, hydrobromides, phosphates, sulphates, hydrogen sulphates, alkylsulphonates, arylsulphonates, acetates, benzoates, citrates, maleates, fumarates, succinates, lactates and tartrates. In addition where the compounds of formula I are sufficiently acidic, pharmaceutically acceptable salts may be formed with an inorganic or organic base which affords a pharmaceutically acceptable cation. Such salts with inorganic or organic bases include for example an alkali metal salt, such as a sodium or potassium salt, an alkaline earth metal salt such as a calcium or magnesium salt, an ammonium salt or for example a salt with methylamine, dimethylamine, trimethylamine, piperidine, morpholine or tris-(2-hydroxyethyl)amine.

Various forms of prodrugs are known in the art. For examples of such prodrug derivatives, see: a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs”, by H. Bundgaard p. 113-191 (1991); c) H. Bundgaard, Advanced Drug Delivery Reviews, 8, 1-38 (1992); d) H. Bundgaard, et al., Journal of Pharmaceutical Sciences, 77, 285 (1988); and e) N. Kakeya, et al., Chem. Pharm. Bull., 32, 692 (1984).

Examples of such pro-drugs may be used to form in-vivo-cleavable esters of a compound of the Formula I. An in-vivo-cleavable ester of a compound of the Formula I containing a carboxy group is, for example, a pharmaceutically-acceptable ester which is cleaved in the human or animal body to produce the parent acid. Suitable pharmaceutically-acceptable esters for carboxy include C₁₋₆alkoxymethyl esters, for example methoxymethyl; C₁₋₆alkanoyloxymethyl esters, for example pivaloyloxymethyl; phthalidyl esters; C₃₋₈cycloalkoxycarbonyloxy C₁₋₆alkyl esters, for example 1-cyclohexylcarbonyloxyethyl; 1,3-dioxolan-2-ylmethyl esters, for example 5-methyl-1,3-dioxolan-2-ylmethyl; and C₁₋₆alkoxycarbonyloxyethyl esters, for example 1-methoxycarbonyloxyethyl; and may be formed at any carboxy group in the compounds of this invention.

Suitable values for R¹, R², R³, R⁴, R⁵, R⁶, R⁷, R⁸, R⁹, R¹⁰ or R¹⁶ or for various substituents on D, R¹², R¹⁴ or R¹⁵ include:
for halogeno fluoro, chloro, bromo and iodo;
for C₁₋₃alkyl: methyl, ethyl, propyl, and isopropyl;
for C₁₋₄alkyl: methyl, ethyl, propyl, isopropyl and tert-butyl;
for N—C₁₋₄alkylamino: methylamino, ethylamino, propylamino, isopropylamino and butylamino;
for N,N-di-(C₁₋₄alkyl)amino: dimethylamino, diethylamino, N-ethyl-N-methylamino and diisopropylamino;
for C₂₋₄alkanoyl: acetyl and propionyl;
for C₂₋₄alkanoylamino: acetamido and propionamido;
for C₁₋₄alkoxy: methoxy and ethoxy;
for C₁₋₃alkoxyC₂₋₃alkyl: methoxyethyl and ethoxypropyl;
for cyanoC₁₋₄alkyl: cyanomethyl and 2-cyanoethyl;
for N—C₁₋₄alkylcarbamoyl: N-methylcarbamoyl, N-ethylcarbamoyl and \overline{N}-propylcarbamoyl;
for N,N-di-(C₁₋₄allyl)carbamoyl: N,N-dimethylcarbamoyl, N-ethyl-N-methylcarbamoyl and N,N-diethylcarbamoyl;
for C₁₋₄alkylsulphonylalkyl: methylsulphonylmethyl and ethylsulphonylmethyl;
for hydroxyC₁₋₄alkyl: hydroxymethyl, 2-hydroxyethyl, 1-hydroxyethyl and 3-hydroxypropyl as appropriate;
for hydroxyC₂₋₃alkyl: 2-hydroxyethyl, 1-hydroxyethyl and 3-hydroxypropyl as appropriate;
for C₁₋₄alkoxyC₁₋₄alkyl: methoxymethyl, ethoxymethyl, 1-methoxyethyl, 2-methoxyethyl, 2-ethoxyethyl and 3-methoxypropyl as appropriate;
for aminoC₁₋₄alkyl or aminomethyl, 2-aminoethyl, 1-aminoethyl and 3-aminopropyl as appropriate;
for aminoC₂₋₃alkyl or 2-aminoethyl, 1-aminoethyl and 3-aminopropyl as appropriate;
for N—C₁₋₄alkylamino₁₋₄alkyl: methylaminomethyl, ethylaminomethyl, 1-methylaminoethyl, 2-methylaminoethyl, 2-ethylaminoethyl and 3-methylaminopropyl as appropriate;
for N,N-di-(C₁₋₄alkyl)aminoC₁₋₄alkyl: dimethylaminomethyl, diethylaminomethyl, 1-dimethylaminoethyl, 2-dimethylaminoethyl and 3-dimethylaminopropyl as appropriate:
for carboxyC₁₋₄alkyl: carboxymethyl, 1-carboxyethyl, 2-carboxyethyl, 3-carboxypropyl and 4-carboxybutyl;
for C₁₋₄alkoxycarbonylC₁₋₄alkyl: methoxycarbonylmethyl, ethoxycarbonylmethyl, tert-butoxycarbonylmethyl, 1-methoxycarbonylethyl, 1-ethoxycarbonylethyl, 2-methoxycarbonylethyl, 2-ethoxycarbonylethyl, 3-methoxycarbonylpropyl and 3-ethoxycarbonylpropyl;
for C₁₋₄alkoxycarbonylamino: methoxycarbonylamino and ethoxycarbonylaamino;
for carbamoylC₁₋₄alkyl: carbamoylmethyl, 1-carbamoylethyl, 2-carbamoylethyl and 3-carbamoylpropyl;

Examples of 5- or 6-membered saturated heterocyclic ring ring systems include pyrrolidinyl, imidazolidinyl, pyrazolidinyl, piperidyl, piperazinyl and morpholinyl. Preferably at least 2 of R¹, R², and R³ are methoxy.

Preferably R¹, R², and R³ are all C₁₋₄alkoxy.

Most preferably R¹, R², and R³ are all methoxy.

Preferably A is ethylene, propylene, benzylene or phenylene. More preferably A is ethylene or phenylene.

More preferably A is phenylene.

Most preferably A is 1,4-phenylene.

Preferably X¹ is —CO—, —CON(R¹⁰)—, —N(R¹⁰)—, —N(R¹⁰)CO— or —OC(O)N(R¹⁰)—.

Most preferably X¹ is —CO— or —N(R¹⁰)CO—.

Preferably R¹⁰ is hydrogen or methyl. Most preferably R¹⁰ is hydrogen.

Preferably Y¹ is propylene or ethylene.

More preferably Y¹ is ethylene.

Preferably B is carboxy sulpho, phosphoryloxy or of the formula —R¹² wherein R¹² is as hereinabove defined.

Yet more preferably B is phosphoryloxy or —R¹². Most preferably, B is —R¹².

Preferably R¹² is a 5 or 6 membered saturated heterocyclic ring containing 1 or 2 ring heteratoms selected from N and O.

Preferably R¹² is a 6 membered saturated heterocyclic ring containing 1 or 2 ring heteratoms selected from N and O.

Preferably —R¹² contains at least 1 ring nitrogen atom.

Preferably —R¹² is piperazinyl, morpholinyl, pyrrolidiyl or piperidinyl, each of which is linked via a ring carbon or nitrogen ring atom and each ring is optionally substituted by 1 or 2 of the substituents mentioned above for —R¹².

Preferably —R¹² is linked via a ring nitrogen atom.

More preferably —R¹² is piperazino or morpholino, each ring being optionally substituted by 1 or 2 of the substituents mentioned hereinabove for —R¹².

The saturated heterocyclic ring may be substituted on ring carbon or ring nitrogen atoms, providing this does not result in quaternisation.

When the saturated heterocyclic ring contains a ring nitrogen atom which is not linked to Y¹, preferably this ring nitrogen atom is substituted.

Preferred substituents for the saturated heterocyclic ring in R¹² include C₁₋₄ alkyl, C₂₋₄alkanoyl, carbamoyl, cyanoC₁₋₃alkyl, hydroxyC₁₋₃alkyl, carboxyC₁₋₃alkyl and aminoC₁₋₃alkyl.

More preferred substituents for the saturated heterocyclic ring in —R¹² include C₁₋₃alkyl, C₂₋₃alkanoyl, carbamoyl and hydroxyC₂₋₃alkyl.

Yet more preferred substituents for the saturated heterocyclic ring in —R¹² include methyl, ethyl, acetyl, propionyl, carbamoyl and 2-hydroxyethyl.

The most preferred substituents for the saturated heterocyclic ring include methyl, acetyl and carbamoyl.

Preferably the saturated heterocyclic ring in —R¹² is unsubstituted or substituted by 1 substituent.

When the saturated heterocyclic ring in —R¹² is morpholino, preferably it is unsubstituted. When the saturated heterocyclic ring in —R¹² is piperazino, preferably it is unsubstituted or substituted by 1 substituent on a ring nitrogen atom.

Most preferably R¹² is morpholino, 4-methylpiperazin-1-yl or 4-acetylpiperazin-1-yl.

Preferably X² is —(CH₂)ᵣ—.

Preferably r is 0, 1 and 2.

Most preferably r is 1.

In another aspect r is 0.

In one aspect the —X²—R¹⁵ substituent is in the 3-position of the phenyl ring in R⁵.

In another aspect the —X²—R¹⁵ substituent is in the 4-position of the phenyl ring in R⁵.

Preferably R¹⁵ is morpholinyl, piperazinyl, piperidinyl or pyrrolidinyl optionally substituted as hereinabove defined for R¹², and substituted by at least 1 substituent selected from C₂₋₄alkanoyl, carbamoyl, N—C₁₋₄-alkylcarbamoyl and N,N-di(C₁₋₄alkyl)carbamoyl.

More preferably R¹⁵ is morpholinyl, piperazinyl or piperidinyl linked by either a ring carbon or nitrogen atom, optionally substituted as hereinabove defined for R¹² and substituted by at least 1 substituent selected from C₂₋₄alkanoyl, carbamoyl, N—C₁₋₄alkylcarbamoyl and N,N-di(C₁₋₄alkyl)carbamoyl.

More preferably, R¹⁵ is morpholino or piperazino, each ring being substituted by 1 substituent selected from C₂₋₃alkanoyl, carbamoyl, N—C₁₋₃alkylcarbamoyl and N,N-di(C₁₋₃alkyl)carbamoyl.

Yet more preferably R¹⁵ is piperazin-1-yl which is substituted in the 4-position by 1 substituent selected from acetyl, carbamoyl, N-methylcarbamoyl and N,N-dimethylcarbamoyl.

Preferably —X²—R¹⁵ is 4-carbamoylpiperazin-1-ylmethyl or 4-acetylpiperazin-1-ylmethyl.

Preferably a is 0, 1, 2 or 3.

More preferably a is 2 or 3.

Most preferably a is 2.

Preferably b is 0, 1, or 2.

More preferably b is 0 or 1

Most preferably b is 0.

Preferably Y² is —C(O)—, —N(R¹⁶)C(O)— or —C(O)N(R¹⁶)—

More preferably Y² is —C(O)— or —N(R¹⁶)C(O)—.

Most preferably Y² is —C(O)—.

Preferably R¹⁶ is hydrogen.

Preferably the alkyl groups in N,N-di(C₁₋₄ alkyl)carbamoylC₁₋₄alkyl in R⁵ are optionally substituted by 1 or 2 substituents selected from amino, N-methylamino, N—N-dimethylamino, hydroxy, methoxy, carboxy, sulpho and phosphoryloxy.

More preferably the alkyl groups in N,N-di(C₁₋₄alkyl)carbamoylC₁₋₄alkyl in R⁵ are optionally substituted by substituents selected from amino, hydroxy and phosphoryloxy.

Yet more preferably the alkyl groups in N,N-di(C₁₋₄alkyl)carbamoylC₁₋₄alkyl are optionally substituted by 1 hydroxy substituent.

In one aspect R⁵ is selected from group 1).

In another aspect R⁵ is selected from group 2).

In yet another aspect R⁵ is selected from group 3).

In yet another aspect R⁵ is selected from group 4).

When R⁵ is selected from group 1), it is preferably 4-[2-(4-methylpiperazin-1-yl)ethylcarbonylamino]phenyl, 4-[2-(4-acetylpiperazin-1-yl)ethylcarbonylamino]phenyl, 4-[2-(4-methylpiperazin-1-yl)methylcarbonylamino]phenyl or 4-[2-(4-acetylpiperazin-1-yl)methylcarbonylamino]phenyl.

When R⁵ is selected from group 2), it is preferably 4-(4-acetylpiperazin-1-ylmethyl)phenyl or 3-(4-acetylpiperazin-1-ylmethyl)phenyl.

When R⁵ is selected from group 3), it is preferably, 2-(4-acetylpiperazin-1-ylcarbonyl)ethyl or 3-(4-acetylpiperazin-1-ylcarbonyl)propyl.

Preferably when R⁵ is selected from group 4), it is N—N-di-(2-hydroxyethyl)carbamoylC₁₋₄alkyl.

Most preferably when R⁵ is selected from group 4), it is 2-[N—N-di-(2-hydroxyethyl)carbamoyl]ethyl or 3-[N,N-di-(2-hydroxyetnyl)carbamoyl]propyl.

Preferably Y³ is —C(O)—, —C(O)O— or —C(O)N(R¹⁸)—. More preferably Y³ is —C(O)— or —C(O)O—.

Most preferably Y³ is —C(O)—.

Preferably R¹⁸ is hydrogen, methyl, 2-hydroxyethyl or 2-aminoethyl.

Most preferably R¹⁸ is hydrogen.

Preferably R¹⁹ is hydrogen or methyl. Most preferably R¹⁹ is hydrogen.

Preferably Y⁴ is —NHCO— or —CONH—.

Preferred optional substituents for alkyl, alkylY⁴ alkyl and phenyl groups in R¹⁷ include: halogeno, amino, N—C₁₋₄alkylamino, N,N-di(C₁₋₄alkyl)amino, C₁₋₄alkoxy, C₁₋₄alkoxycarbonylamino, C₁₋₄ alkanoyl, phosphoryloxy, R¹² (wherein R¹² is as hereinabove defined), —Y⁵R²⁰ [wherein Y⁵ is —NHCO—; and R²⁰ is C₁₋₄alkyl or R²² (wherein R²² is as hereinabove defined)].

More preferred optional substituents for alkyl, alkylY⁴alkyl and phenyl groups include: fluoro, chloro, bromo, amino, methoxy, methoxycarbonylamino, acetyl, phosphoryloxy, R¹² (wherein R¹² is as hereinabove defined), —Y⁵R²⁰ [wherein Y⁵ is —NHCO—; and R²⁰ is methyl, ethyl or R²² (wherein R²² is as hereinabove and hereinbelow defined)].

Yet more preferably, optional substituents for alkyl, alkylY⁴ alkyl and phenyl groups in R¹⁷ include fluoro, chloro and bromo. Most preferably alkyl and alkylY⁴ alkyl groups in R¹⁷ are unsubstituted.

Preferably R²¹ is hydrogen.

Preferably R²² is optionally substituted: imidazolyl, pyridyl, pyrimidyl, thiazolyl or pyrazinyl.

More preferably R²² is optionally substituted: imidazolyl.

A preferred optional substituent for the aromatic heterocyclic group in R²² is C₁₋₄alkyl.

A more preferred optional substituent for the aromatic heterocyclic group in R²² methyl.

The aromatic heterocyclic group in R²² may be unsubstituted.

Preferably R²³ and R²⁴ are independently hydrogen or methyl.

More preferably R²³ and R²⁴ are hydrogen.

Preferably R²⁵ and R²⁶ are independently selected from hydrogen and methyl. More

Preferably R²⁵ and R²⁶ are hydrogen.

Preferably R²⁷ is C₁₋₃alkyl.

More preferably R²⁷ is methyl.

Preferably R²²—C₁₋₄ alkyl in group 3) of R⁸ is R²²-methylene, R²²-propylene.

More preferably R²²—C₁₋₄ alkyl is R²²-ethylene.

Preferably Y⁷ is —N(R²³)C(O)— or —CON(R²³)—.

More preferably Y⁷ is —N(R²³)C(O)— or —CON(R²³)—.

More preferably Y⁷ is —NHC(O)— or —CONH—.

Preferably R¹⁷ is methyl, fluoromethyl, difluoromethyl or trifluoromethyl.

More preferably R¹⁷ is methyl.

Most preferably R⁸ is acetyl.

Most preferably R⁹ is hydrogen.

A preferred class of compound is of the formula (I) wherein:

R¹, R², and R³ are all C₁₋₄alkoxy;

R⁴ and R⁶ are independently selected from hydrogen, hydroxy, C₁₋₃ alkoxy, and C₁₋₃alkyl;

R⁵ is selected from one of the following groups:

1) of the formula -A-X¹—Y¹—B, wherein:

A is ethylene or phenylene;

Y¹ is C₁₋₃alkylene;

X¹ is —CO—, —CON(R¹⁰)—, —N(R¹⁰)—, —N(R¹⁰)CO— or —OC(O)N(R¹⁰)—;

B is carboxy sulpho, phosphoryloxy or of the formula —R¹² (wherein R¹² is piperazinyl, morpholinyl or piperidinyl, each of which is linked via a ring carbon or nitrogen ring atom and each ring is optionally substituted by 1 or 2 substituents selected from C₁₋₄alkyl, C₂₋₄alkanoyl, carbamoyl, cyanoC₁₋₃alkyl, hydroxyC₁₋₃alkyl, carboxyC₁₋₃alkyl and aminoC₁₋₃alkyl);

2) of the formula 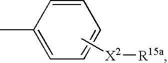
wherein:

the —X²—R^{15a} substituent is in the 3, or 4-position of the phenyl ring;

X² is —(CH₂)ᵣ—;

r is 0, 1 and 2; and

R^{15a} is morpholinyl, piperazinyl, piperidinyl or pyrrolidinyl optionally substituted by 1 or 2 substituents selected from C₁₋₄alkyl, C₂₋₄alkanoyl, carbamoyl, cyanoC₁₋₃alkyl, hydroxyC₁₋₃alkyl, carboxyC₁₋₃alkyl and aminoC₁₋₃alkyl; and substituted by at least 1 substituent selected from C₂₋₄alkanoyl, carbamoyl, N—C₁₋₄ alkylcarbamoyl and N,N-di(C₁₋₄alkyl)carbamoyl;

3) of the formula —(CH₂)ₐ—Y²—(CH₂)_{b}—R^{15b}, wherein:

a is 2 or 3;

b is 0, 1, or 2; and

Y² is a single direct bond, —C(O)—, —NHC(O)— or —C(O)NH—; and

R^{15b} is morpholinyl, piperazinyl, piperidinyl or pyrrolidinyl optionally substituted by 1 or 2 substituents selected from C₁₋₄alkyl, C₂₋₄alkanoyl, carbamoyl, cyanoC₁₋₃alkyl, hydroxyC₁₋₃alkyl, carboxyC₁₋₃alkyl and aminoC₁₋₃alkyl; and substituted by at least 1 substituent selected from C₂₋₄alkanoyl, carbamoyl, N—C₁₋₄ alkylcarbamoyl and N,N-di(C₁₋₄alkyl)carbamoyl; or

4) N,N-di(C₁₋₄alkyl)carbamoylC₁₋₄alkyl-, wherein the alkyl group is optionally substituted by 1 or 2 substituents selected from amino, N-methylamino, N—N-dimethylamino, hydroxy, methoxy, carboxy, sulpho and phosphoryloxy; and

R⁸ is a group —Y³R¹⁷ (wherein Y³ is —C(O)—, —C(O)O— or —C(O)NH—; and R¹⁷ is selected from one of the following 4 groups:

1) hydrogen, C₁₋₄alkyl, phenyl or C₁₋₄alkylY⁴C₁₋₄alkyl (wherein Y⁴ is —NHCO— or —CONH—); [which alkyl, alkylY⁴alkyl or phenyl group is optionally substituted by 1 or 2 substituents selected from:

halogeno, amino, N—C₁₋₄alkylamino, N,N-di(C₁₋₄alkyl)amino, C₁₋₄alkoxy, C₁₋₄alkoxycarbonylamino, C₁₋₄alkanoyl, phosphoryloxy, R^{12a} (wherein R^{12a} is piperazinyl, morpholinyl or piperidinyl, each of which is linked via a ring carbon or nitrogen ring atom and each ring is optionally substituted by 1 or 2 of the substituents selected from C₁₋₄alkyl, C₂₋₄alkanoyl, carbamoyl, cyanoC₁₋₃alkyl, hydroxyC₁₋₃alkyl, carboxyC₁₋₃alkyl and aminoC₁₋₃alkyl), —Y⁵—R²⁰ [wherein Y⁵ is —NHCO—; and R²⁰ is C₁₋₄alkyl or R^{22a} (wherein R^{22a} is imidazolyl, pyridyl, pyrimidyl, thiazolyl or pyrazinyl, each of which is optionally substituted by C₁₋₄alkyl)];

2) R^{22a} (wherein R^{22a} is as hereinabove defined);

3) R^{22a}—C₁₋₄alkyl- (wherein R^{22a} is as hereinabove defined); or

4) R^{12a}Y⁷C₁₋₄alkyl- (wherein R^{12a} is as hereinabove defined and Y⁷ is Y⁷ is —NHC(O)— or —CONH—)];

and R⁹ is hydrogen;

or a pharmaceutically-aceeptable salt, solvate or pro-drug thereof.

Another preferred class of compound is of the formula (1) wherein:

R¹, R², and R³ are all methoxy;

R⁴ and R⁶ are independently selected from hydrogen, hydroxy, methoxy and methyl;

R⁵ is selected from one of the following groups:

1) of the formula -A-X¹—Y¹—B, wherein:

A is ethylene or phenylene;

Y¹ is C₁₋₃alkylene;

X¹ is —CO—, —CON(R¹⁰)—, —N(R¹⁰)—, —N(R¹⁰)CO— or —OC(O)N(R¹⁰)—;

B is carboxy sulpho, phosphoryloxy or of the formula —R¹² (wherein R¹² is

piperazinyl, morpholinyl or piperidinyl, each of which is linked via a ring carbon or nitrogen ring atom and each ring is optionally substituted by 1 or 2 substituents selected from C₁₋₄ alkyl, C₂₋₄alkanoyl, carbamoyl, cyanoC₁₋₃alkyl, hydroxyC₁₋₃alkyl, carboxyC₁₋₃alkyl and aminoC₁₋₃alkyl);

2) of the formula 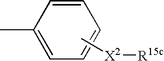
wherein:

the —X²—R^{15c} substituent is in the 3, or 4-position of the phenyl ring;

X² is —(CH₂)ᵣ—;

r is 0, 1 and 2; and

R^{15c} is morpholinyl, piperazinyl or piperidinyl optionally substituted by 1 or 2 substituents selected from C₁₋₄ alkyl, C₂₋₄alkanoyl, carbamoyl, cyanoC₁₋₃alkyl, hydroxyC₁₋₃alkyl, carboxyC₁₋₃alkyl and aminoC₁₋₃alkyl; and substituted by at least 1 substituent selected from C₂₋₄alkanoyl, carbamoyl, N—C₁₋₄ alkylcarbamoyl and N,N-di(C₁₋₄alkyl)carbamoyl;

3) of the formula —(CH₂)ₐ—Y²—(CH₂)_{b}—R^{15d}, wherein:

a is 2 or 3;

b is 0 or 1; and

Y² is a single direct bond, —C(O)— or —NHC(O)—; and

R^{15d} is morpholinyl, piperazinyl or piperidinyl optionally substituted by 1 or 2 substituents selected from C₁₋₄ alkyl, C₂₋₄alkanoyl, carbamoyl, cyanoC₁₋₃alkyl, hydroxyC₁₋₃alkyl, carboxyC₁₋₃alkyl and aminoC₁₋₃alkyl; and substituted by at least 1 substituent selected from C₂₋₄alkanoyl, carbamoyl, N—C₁₋₄ alkylcarbamoyl and N,N-di(C₁₋₄alkyl)carbamoyl; or

4) N,N-di(C₁₋₄alkyl)carbamoylC₁₋₄alkyl-, wherein the alkyl group is optionally substituted by 1 or 2 substituents selected from amino, N-methylamino, N—N-dimethylamino, hydroxy, methoxy, carboxy, sulpho and phosphoryloxy; and

R⁸ is a group —Y³R¹⁷ (wherein Y³ is —C(O)— or —C(O)O—; and R¹⁷ is selected from one of the following 4 groups:

1) C₁₋₄alkyl [which alkyl, group is optionally substituted by 1 or 2 substituents selected from: fluoro, chloro and bromo;

2) R^{22b} (wherein R^{22b} is imidazolyl, pyridyl, pyrimidyl, thiazolyl or pyrazinyl, each of which is optionally substituted by C₁₋₄alkyl);

3) R^{22b}—C₁₋₄alkyl- (wherein R^{22b} is as hereinabove defined); or

4) R^{12b}Y⁷C₁₋₄alkyl- (wherein R^{12b} is morpholinyl, piperidinyl or piperazinyl each of which is optionally substituted by methyl, ethyl, acetyl, propionyl, carbamoyl or 2-hydroxyethyl; and Y⁷ is —NHC(O)— or —CONH—)];

and R⁹ is hydrogen;

or a pharmaceutically-aceeptable salt, solvate or pro-drug thereof.

A preferred compound of the present invention is of the formula (II):

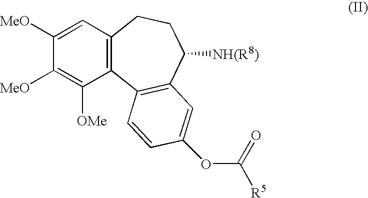

wherein R⁵ and R⁸ are as hereinabove defined;

or a pharmacetically-acceptable salt, solvate or pro-drug thereof.

Another preferred class of compounds is that of the formula (III) wherein:

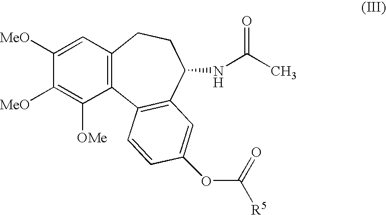

R⁵ is selected from one of the following groups:

1) of the formula -A-X¹—Y¹—B, wherein:

A is ethylene or phenylene;

Y¹ is C₁₋₃alkylene;

X¹ is —CO—, —CONH—, —NH—, —NHCO— or —OC(O)NH—;

B is carboxy sulpho, phosphoryloxy or of the formula —R¹² (wherein R¹² is

piperazinyl, morpholinyl or piperidinyl, each of which is linked via a ring carbon or nitrogen ring atom and each ring is optionally substituted by 1 or 2 of the substituents selected from C₁₋₄ alkyl, C₂₋₄alkanoyl, carbamoyl, cyanoC₁₋₃alkyl, hydroxyC₁₋₃alkyl, carboxyC₁₋₃alkyl and aminoC₁₋₃alkyl);

2) of the formula

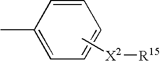

wherein:

the —X²—R¹⁵ substituent is in the 3, or 4-position of the phenyl ring;

X² is —(CH₂)ᵣ—;

r is 0, 1 and 2; and

R¹⁵ is morpholinyl, piperazinyl, piperidinyl or pyrrolidinyl optionally substituted as immediately hereinabove defined for R¹², and substituted by at least 1 substituent selected from C₂₋₄alkanoyl, carbamoyl, N—C₁₋₄ alkylcarbamoyl and N,N-di(C₁₋₄alkyl)carbamoyl;

3) of the formula —(CH₂)ₐ—Y²—(CH₂)_{b}—R¹⁵, wherein:

a is 2 or 3;

b is 0, 1, or 2; and

Y² is a single direct bond, —C(O)—, —NHC(O)— or —C(O)NH—; or

4) N,N-di(C₁₋₄alkyl)carbamoylC₁₋₄alkyl-, wherein the alkyl group is optionally substituted by 1 or 2 substituents selected from amino, N-methylamino, N—N-dimethylamino, hydroxy, methoxy, carboxy, sulpho and phosphoryloxy;

or a pharmaceutically acceptable salt, solvate or prodrug thereof.

Another preferred class of compounds is that of the formula (III) wherein:

R⁵ is selected from one of the following groups:

1) of the formula -A-X¹—Y¹—B, wherein:

A is ethylene or phenylene;

Y¹ is C₁₋₃alkylene;

X¹ is —CO—, —NHCO—;

B is carboxy sulpho, phosphoryloxy or of the formula —R¹² (wherein R¹² is

piperazinyl, morpholinyl or piperidinyl, each of which is linked via a ring carbon or nitrogen ring atom and each ring is optionally substituted by 1 or 2 of the substituents selected from C₁₋₄ alkyl, C₂₋₄alkanoyl, carbamoyl, cyanoC₁₋₃alkyl, hydroxyC₁₋₃alkyl, carboxyC₁₋₃alkyl and aminoC₁₋₃alkyl);

2) of the formula

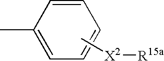

wherein:

the —X²—R^{15a} substituent is in the 3, or 4-position of the phenyl ring;

X² is —(CH₂)ᵣ—;

r is 1 and 2; and

R^{15a} is as hereinabove defined;

3) of the formula —(CH₂)ₐ—Y²—(CH₂)_{b}—R^{15b}, wherein:

a is 2 or 3;

b is 0; and

Y² is a single direct bond, —C(O)—, —NHC(O)— or —C(O)NH—; and

R^{15b} is as hereinabove defined; or

4) N,N-di(C₁₋₄alkyl)carbamoylC₁₋₄alkyl-, wherein the alkyl group is optionally substituted by 1 or 2 substituents selected from amino, hydroxy and phosphoryloxy; or a pharmaceutically acceptable salt, solvate or pro-drug thereof.

Another preferred class of compounds is that of the formula (III) wherein:

R⁵ is selected from one of the following groups:

1) of the formula -A-X¹—Y¹—B, wherein:

A is phenylene;

X¹ is —CO—, —NHCO—;

Y¹ is methylene or ethylene;

B is piperazino or morpholinyl each of which is linked via a ring nitrogen atom and each ring is optionally substituted by 1 methyl or acetyl group;

2) of the formula

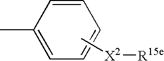

wherein:

the —X²—R^{15e} substituent is in the 3, or 4-position of the phenyl ring;

X² is —(CH₂)ᵣ—;

r is 1; and

R^{15e} is piperazino or morpholinyl each of which is linked via a ring nitrogen atom and each ring is optionally substituted by 1 methyl or acetyl group;

3) of the formula —(CH₂)ₐ—Y²—(CH₂)_{b}—R^{15f}, wherein:

a is 2 or 3;

b is 0; and

Y² is —C(O)—;

R^{15f} is piperazino or morpholinyl each of which is linked via a ring nitrogen atom and each ring is optionally substituted by 1 methyl or acetyl group; or

4) 2-[N,N-di(C₁₋₄alkyl)carbamoyl]ethyl- or 3-[N,N-di(C₁₋₄alkyl)carbamoyl]propyl-, wherein the C₁₋₄alkyl group is optionally substituted by 1 hydroxy group;

or a pharmaceutically acceptable salt, solvate or prodrug thereof.

Yet another preferred class of compounds is that of the formula (III) wherein:

R⁵ is 2-[N,N-di(C₁₋₄alkyl)carbamoyl]ethyl- or 3-[N,N-di(C₁₋₄alkyl)carbamoyl]propyl-, wherein the C₁₋₄alkyl group is optionally substituted by 1 hydroxy group; or a pharmaceutically acceptable salt, solvate or prodrug thereof. Particular compounds of the present invention include: (5S)-5-acetylamino-9,10,11-trimethoxy-6,7-dihydro-5H-dibenzo[a,c]cyclohepten-3-yl 5-(4-acetylpiperazin-1-yl)-5-oxopentanoate; (5S)-5-acetylamino-9,10,11 -trimethoxy-6,7-dihydro-5H-dibenzo[a,c]cyclohepten-3-yl 4-(4-acetylpiperazin-1-yl)-4-oxobutanoate; (5S)-5-acetylamino-9,10,11-trimethoxy-6,7-dihydro-5H-dibenzo[a,c]cyclohepten-3-yl 3-(4-acetylpiperazin-1-ylmethyl)benzoate; (5S)-5-acetylamino-9,10,11-trimethoxy-6,7-dihydro-5H-dibenzo[a,c]cyclohepten-3-yl 4-[3-(4-methylpiperazin-1-yl)propionylamino]benzoate; (5S)-5-acetylamino-9,10,11 -trimethoxy-6,7-dihydro-5H-dibenzo[a,c]cyclohepten-3-yl 3-(4-carbamoylpiperazin-1-ylmethyl)benzoate; (5S)-5-acetylamino-9,10,11-trimethoxy-6,7-dihydro-5H-dibenzo[a,c]cyclohepten-3-yl N-acetylpiperidin-1-ylcarboxylate; (5S)-5-acetylamino-9,10,11-trimethoxy-6,7-dihydro-5H-dibenzo[a,c]cyclohepten-3-yl 3-[N,N-di-(2-hydroxyethyl)carbamoyl]propanoate; and (5S)-5-acetylamino-9,10,11-trimethoxy-6,7-dihydro-5H-dibenzo[a,c]cyclohepten-3-yl 4-[N,N-di(2-hydroxyethyl)carbamoyl]butanoate; and pharmaceutically-acceptable salts, solvates or pro-drugs thereof.

Synthesis of Compounds of the Formula I

Compounds of Formula I may be prepared by a number of processes as generally described herein below and more specifically in the Examples hereinafter. Processes for the preparation of novel compounds of formula I, are provided as a further feature of the invention and are as described hereinafter. Necessary starting materials may be obtained by standard procedures of organic chemistry. The preparation of such starting materials is described within the accompanying non-limiting Examples. Alternatively necessary starting materials are obtainable by analogous procedures to those illustrated which are within the ordinary skill of an organic chemist.

Thus according to another aspect of the invention, a compound of the formula (I) may be formed by deprotecting a compound of the formula (I) wherein at least 1 functional group is protected. For example, amino, hydroxy, carboxy or phosphoryloxy groups may be protected during the reaction sequence used to prepare a compound of the formula (1).

Protecting groups may in general be chosen from any of the groups described in the literature or known to the skilled chemist as appropriate for the protection of the group in question, and may be introduced by conventional methods.

Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

A suitable protecting group for a hydroxy group is, for example, an arylmethyl group (especially benzyl), a tri-(1-4C)alkylsilyl group (especially trimethylsilyl or tert-butyldimethylsilyl), an aryldi-(1-4C)alkylsilyl group (especially dimethylphenylsilyl), a diaryl-(1-4C)alkylsilyl group (especially tert-butyldiphenylsilyl), a (1-4C)alkyl group (especially methyl), a (2-4C)alkenyl group (especially allyl), a (1-4C)alkoxymethyl group (especially methoxymethyl) or a tetrahydropyranyl group (especially tetrahydroyran-2-yl). The deprotection conditions for the above protecting groups will necessarily vary with the choice of protecting group. Thus, for example, an arylmethyl group such as a benzyl group may be removed, for example, by hydrogenation over a catalyst such as palladium-on-charcoal. Alternatively a trialkylsilyl or an aryldialkylsilyl group such as a tert-butyldimethylsilyl or a dimethylphenylsilyl group may be removed, for example, by treatment with a suitable acid such as hydrochloric, sulphuric, phosphoric or trifluoroacetic acid, or with an alkali metal or ammonium fluoride such as sodium fluoride or, preferably, tetrabutylammonium fluoride. Alternatively an alkyl group may be removed, for example, by treatment with an alkali metal (1-4C)alkylsulphide such as sodium thioethoxide or, for example, by treatment with an alkali metal diarylphosphide such as lithium diphenylphosphide or, for example, by treatment with a boron or aluminium trihalide such as boron tribromide. Alternatively a (1-4C)alkoxymethyl group or tetrahydropyranyl group may be removed, for example, by treatment with a suitable acid such as hydrochloric or trifluoroacetic acid.

Alternatively a suitable protecting group for a hydroxy group is, for example, an acyl group, for example a (2-4C)alkanoyl group (especially acetyl) or an aroyl group (especially benzoyl). The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide.

A suitable protecting group for an amino, imino or alkylamino group is, for example, an acyl group, for example a (2-4C)alkanoyl group (especially acetyl), a (1-4C)alkoxycarbonyl group (especially methoxycarbonyl, ethoxycarbonyl or tert-butoxycarbonyl), an arylmethoxycarbonyl group (especially benzyloxycarbonyl) or an aroyl group (especially benzoyl). The deprotection conditions for the above protecting groups necessarily vary with the choice of protecting group. Thus, for example, an acyl group such as an alkanoyl, alkoxycarbonyl or aroyl group may be removed for example, by hydrolysis with a suitable base such as an alkali metal hydroxide, for example lithium or sodium hydroxide. Alternatively an acyl group such as a tert-butoxycarbonyl group may be removed, for example, by treatment with a suitable acid such as hydrochloric, sulphuric or phosphoric acid or trifluoroacetic acid, and an arylmethoxycarbonyl group such as a benzyloxycarbonyl group may be removed, for example, by hydrogenation over a catalyst such as palladium-on-charcoal.

A suitable protecting group for a carboxy group is, for example, an esterifying group, for example a (1-4C)alkyl group (especially methyl or ethyl) which may be removed, for example, by hydrolysis with a suitable base such as an alkali metal hydroxide, for example lithium or sodium hydroxide; or, for example, a tert-butyl group which may be removed, for example, by treatment with a suitable acid such as hydrochloric, sulphuric or phosphoric acid or trifluoroacetic acid.

In the following process description (the symbols R¹—R⁷, A, B, D, R^{a}, R^{b}, a and b are to be understood to represent those groups described above in relation to formulae (I), (II) and (III) unless otherwise stated.

A compound of the formula (I), or a compound of the formula (I) wherein at least 1 functional group is protected, may be prepared using one of the following processes:

(a) reacting a compound of the formula (X):

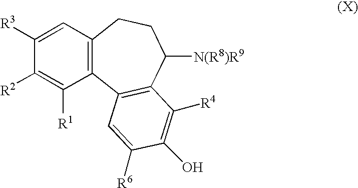

with a compound of the formula R⁵—COOH or an activated derivative thereof;

(b) when R⁵ is of the formula:

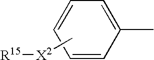

reacting a compound of the formula (XI):

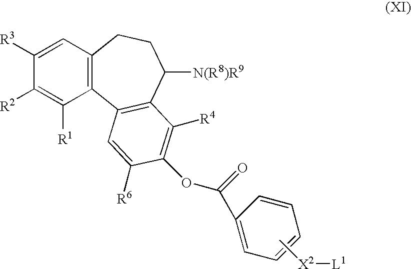

with R¹⁵ (wherein L¹ is a leaving group);

(c) introducing substituents onto a ring nitrogen atom in R¹² or R¹⁵;

(d) converting one compound of the formula (I) into another compound of the formula (I);

(e) when a phosphoryloxy group is desired, reacting the corresponding hydroxy compound with a phosphoramidite;

wherein any functional groups are optionally protected.

and thereafter if necessary:

i) converting a compound of formula (I) into another compound of formula (I);

ii) removing any protecting groups;

iii) forming a pharmaceutically acceptable salt, solvate or pro-drug thereof.

The reaction between a compound of the formula (X) and a compound of the formula R⁵—COOH or an activated derivative thereof is performed under standard coupling conditions. For example, in the presence of a coupling agent such as dicyclohexylcarbodiimide or 1-(3-dimethylaminopropyl)-3-ethylcarbodiimide, and optionally a base, for example an organic base such as triethylamine or DMAP. The reaction is conveniently carried out in a solvent such as an aprotic solvent, for example dimethylformamide, or in a chlorinated solvent, for example trichloromethane or dichloromethane, and at a temperature in the range of about −30° C. to about 60° C. Conveniently at or near ambient temperature.

When X² is —(CH₂)r, the reaction between a compound of the formula (XI) and R¹⁵ is conveniently carried out in an inert organic solvent such as acetonitrile, in a temperature range of 0° C. to 60° C., normally at ambient temperature. Suitable leaving groups (L¹) include halogeno, mesyloxy and tosyloxy. Preferably halogeno, and particularly chloro or iodo.

When X² is —CO—, L¹ is usually chloro and the reaction is normally carried out in a chlorinated solvent such as dichloromethane. The reaction is carried out in the presence of a base such as triethylamine and in a temperature range of 0 to 60° C., normally about ambient temperature.

Substituents, such as C₁₋₄alkyl, C₂₋₄alkanoyl, carbamoyl and alkylated carbamoyl groups, can be in introduced onto a ring nitrogen atom in R¹² or R¹⁵, using standard conditions known in the art for alkylation and acylation of an amino group. Alkylations are normally carried out by reacting a ring-nitrogen containing R¹² or R¹⁵ with the appropriate alkylating agent, such as an alkyl halide, an alkyl toluenesulphonate, an alkyl methanesulphonate or an alkyl triflate. The alkylation reaction can be carried out in the presence of a base for example an inorganic base such as a carbonate e.g. caesium or potassium carbonate, a hydride such as sodium hydride or an alkoxide such as potassium tert-butoxide in a suitable solvent such as an aprotic solvent e.g. dimethylformamide or an ether solvent such as tetrahydrofuran at a temperature of around −10° C. to 80° C.

Acylation of a ring nitrogen in R¹² or R¹⁵ is carried out by reacting the saturated heterocyclic ring with an acylating agent, for example, an acyl halide or anhydride in the presence of a base, for example a tertiary amine base such as triethylamine, in for example, a solvent such as a hydrocarbon solvent e.g. dichloromethane, at a temperature in the range for example −30° C. to 120° C., conveniently at or near ambient temperature.

A carbamoyl group can be introduced by reacting the saturated heterocyclic ring with a tri(alkyl)silyl isocyanate in an inert organic solvent such as dichloromethane.

A compound of formula I may also be prepared from another compound of formula I by chemical modification. Examples of such chemical modifications include standard alkylation, arylation, heteroarylation, acylation, sulphonylation, phosphorylation, aromatic halogenation and coupling reactions. These reactions may be used to add new substituents or to modify existing substituents. Alternatively, existing substituents in compounds of formula I may be modified by, for example, oxidation, reduction, elimination, hydrolysis or other cleavage reaction to yield other compounds of formula I. For example a substituent can be introduced onto a ring nitrogen atom in R¹² using similar processes to those described above, for the alkylation or acylation of a ring nitrogen.

In another general example an alkoxy group may be cleaved to the corresponding hydroxy group by reaction with boron tribromide, in a solvent such as a chlorinated solvent e.g. dichloromethane, at a low temperature e.g. around −78° C.

A amino group can be alkylated or acylated using similar reaction conditions to those described above for alkylation or acylation of a ring nitrogen atom in R¹² or R¹⁵.

A compound containing a hydroxy group can be converted into the corresponding phosphoryloxy compound by treatment with for example di-tert-butyl diisopropylphoramidite or di-tert-butyl diethylphosphoramidite, in the presence of a suitable catalyst for example tetrazole. A solvent, such as an ether solvent, for example tetrahydrofuran can be used at a temperature in the range of −40° C. to 40° C., conveniently at or near ambient temperature, followed by treatment with an oxidising agent, such as 3-chloroperoxy benzoic acid. The reaction is carried out at a temperature in the range −78° C. to 40° C., preferably −40° C. to 10° C. The resulting intermediate phosphate triester is treated with an acid for example trifluoroacetic acid in a solvent such as a chlorinated solvent e.g. dichloromethane at a temperature in the range −30° C. to 40° C. conveniently at or near 0° C. to give the phosphoryloxy compound.

Synthesis of Intermediates

A compound of the formula (X) may be known in the art or may be prepared from by (f) reacting a compound of the formula (XII):

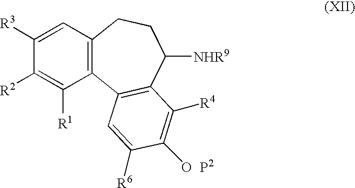

wherein P² is an hydroxy-protecting group, with a compound of the formula L²-R⁸, wherein L² is a leaving group;

The reaction between a compound of the formula (XII) and a compound of the formula L²-R⁸ is conveniently performed under standard acylation or sulphonylation conditions. L¹ is usually halogeno, for example chloro or bromo, hydroxy, mesyloxy or tosyloxy or an ‘activated’ hydroxy group. The precise conditions depending largely upon the nature of R⁸.

For example, when Y³ is —CO—, L² may be hydroxy and the reaction is normally carried out in the presence of coupling agent such as dicyclohexylcarbodiimide or 1-(3-dimethylaminopropyl)-3-ethylcarbodiimide. Optionally, a base may be used, for example an organic base such as triethylamine. Suitable solvents are usually aprotic solvents, for example dimethylformamide, or chlorinated solvents, for example trichloromethane or dichloromethane. The temperature is usually in the range of about −30° C. to about 60° C., conveniently at or near ambient temperature.

When Y³ is —C(O)O—, L² is usually an ‘activated’ hydroxy group. That is a group which acts as a leaving group in the same way as hydroxy, but is more labile. It can be formed in situ. An example, of an activated hydroxy group is 4-nitrophenoxy, in which case the compound R⁸-L² can be formed by reacting a hydroxy group (R¹⁷—OH) with 4-nitrophenylchloroformate. The reaction is usually carried out in an organic solvent such as dichloromethane, acetonitrile or tetrahydrofuran, in a temperature range of about −20° C. to the reflux temperature of the solvent. In addition an organic base such as triethylamine or N-methylmorpholine is normally present. Alternatively, a compound of the formula (XII) can be reacted with 4-nitrophenylchloroformate and the resulting intermediate reacted with R¹⁷—OH under similar conditions to those described above for the reaction of a compound of the formula (XII) with a compound of the formula R⁸-L² wherein L² is 4-nitrophenoxy.

When Y³ is —CON(R¹⁸)—, L² is preferably halogeno, particularly chloro. Alternatively when -A- is —CONH—, a compound of the formula (XII) can be reacted with an isocyanate of the formula C≡N—R¹⁷. These reactions are conveniently carried out in the presence of a base, particularly an organic base, such as triethylamine, pyridine or N-methylmorpholine, triethylamine, pyridine or N-methylmorpholine in a solvent such as an ether solvent for example tetrahydrofuran or in a chlorinated solvent for example dichloromethane at a temperature in the range from about —20° C. to the reflux temperature of the solvent. Alternatively, a compound of the formula (XII) can be reacted with 4-nitrophenylchloroformate and the resulting intermediate reacted with R¹⁷—NH₂ under similar conditions to those described above for the reaction of a compound of the formula (XII) with a compound of the formula R⁸-L² wherein L² is 4-nitrophenoxy.

When —X¹— is of the formula —SO₂N(R⁸)—, L² is preferably halogeno, for example chloro. The reaction is conveniently carried out in the presence of a base such as dimethylaniline, in a chlorinated solvent such as trichloromethane and at a temperature in the range from about −20° C. to about 60° C. More preferably in pyridine, at a temperature in the range from about −20° C. to about 60° C. A compound of the formula (XI) may be prepared by reacting a compound of the formula (X) with a compound of L¹-X²-phenyl-COOH in which L¹ is protected or a precursor of L¹ is used, using similar conditions to those described for the formation of a compound of the formula (I) from a compound of the formula (X).

L¹ can be deprotected or the precursor converted to L¹ following the coupling with the compound of the formula (X).

A compound of the formula (XII) can be formed from a compound of the formula (X) or (XI) wherein R⁸ is hydrogen, using similar conditions to those described above for the formation of a compound of the formula (I).

Compounds of the formula R⁵—COOH may be known in the art or prepared by methods known in the art or analogous to those illustrated in the specific examples. The following description gives some general processes for preparing some compounds of the formula R⁵—COOH.

When R⁵ is of the formula -A-X¹Y¹—B, and X¹ is —N(R¹⁰)CO— or —CON(R¹⁰)— then a compound of the formula R⁵—COOH can be formed by reacting a compound of the formula P¹OOC-A-NHR¹⁹, wherein P¹ is a carboxy-protecting group, with a compound of the formula HOOC—Y¹—B, or of the formula P¹OOC-A-COOH with a compound of the formula NH(R¹⁰)—Y¹—B, under standard amide forming conditions. Similarly when X¹ is of the formula —N(R¹⁰)SO²— or SO²N(R¹⁰)— a compound of the formula R⁵—COOH can be formed by reacting the appropriate amine and sulphonyl chloride under conditions known for the formation of a sulphonamide. Likewise a compound of the formula R⁵—COOH wherein X¹ is —C(O)O— can be formed by reacting together the appropriate carboxylic acid and alcohol and when X¹ is —N(R¹⁰)C(O)O—, by reacting together the appropriate amine and ROC(O)OR compound.

When R⁵ is of the formula R¹⁵—X²-phenyl, a compound of the formula R⁵—COOH can be formed by reacting together the appropriate L¹-(CH₂)ᵣ— or L¹-CO-substituted benzoic acid, wherein the carboxylic group in the benzoic acid is protected during the course of the reaction, under similar conditions to that described for the reaction between a compound of the formula (XI) with R¹⁵.

When R⁵ is of the formula —(CH₂)a-Y²—(CH₂)b and —R¹⁵ a is 2 or 3 and b is 0, a compound of the formula R⁵—COOH can be formed by reacting R¹⁵ with succinic anhydride or glutaric anhydride, as appropriate. The reaction is normally carried out in an inert organic solvent such as dichloromethane, in a temperature range of 0° to 60° C., usually around ambient temperature.

When R⁵ is N,N-di-(C₁₋₄alkyl)carbamoylC₁₋₄alkyl and C₁₋₄alkyl is ethyl or propyl, a compound of the formula R⁵—COOH can be formed by reacting the HN(C₁₋₄alkyl)₂ compound with succinic anhydride or glutaric anhydride as appropriate. The reaction is normally carried out in an inert organic solvent such as dichloromethane, in a temperature range of 0° to 60° C., usually around ambient temperature.

Acid addition salts of the compounds of formula I are prepared in a conventional manner by treating a solution or suspension of the free base I with about one equivalent of a pharmaceutically acceptable acid. Salts of compounds of formula I derived from inorganic or organic bases are prepared in a conventional manner by treating a solution or suspension of the free acid I with about one equivalent of a pharmaceutically acceptable organic or inorganic base. Alternatively both acid addition salts and salts derived from bases may be prepared by treatment of the parent compound with the appropriate ion-exchange resin in a standard fashion. Conventional concentration and recrystallistion techniques are employed in isolating the salts.

Compounds according to the invention are able to destroy vasculature that has been newly formed such as tumour vasculature while leaving unaffected normal, mature vasculature. The identification of compounds which selectively, and preferably potently, damage newly-formed vasculature is desirable and is the subject of the present invention. The ability of the compounds to act in this way may be assessed, for example, using one or more of the procedures set out below:

(a) Activity Against Tumour Vasculature Measured by Radioactive Tracer

This assay demonstrates the ability of compounds to damage selectively tumour vasculature.

Subcutaneous CaNT tumours were initiated by injecting 0.05 ml of a crude tumour cell suspension, approximately 10⁶ cells, under the skin overlying the rear dorsum of 12-16 week-old mice. The animals were selected for treatment after approximately 3-4 weeks, when their tumours reached a geometric mean diameter of 5.5-6.5 mm. Compounds were dissolved in sterile saline and injected intraperitoneally in a volume of 0.1 ml per 10 g body weight. Tumour perfusion was measured 6 hours after intraperitoneal administration in tumour, kidney, liver, skin, muscle, gut and brain by the ⁸⁶RbCl extraction technique (Sapirstein, Amer. Jnl. Physiol., 1958, 193, 161-168). Tissue radioactivity measured 1 minute after an intravenous injection of ⁸⁶RbCl was used to calculate relative blood flow as a proportion of cardiac output (Hill and Denekamp, Brit. Jnl. Radiol., 1982, 55, 905-913). Five animals were used in control and treated groups. Results were expressed as a percentage of the blood flow in the corresponding tissues in vehicle treated animals.

(b) Activity Against Tumour Vasculature Measured by Fluorescent Dye

This assay demonstrates the ability of compounds to damage tumour vasculature.

Tumour functional vascular volume in CaNT tumour-bearing mice was measured using the fluorescent dye Hoechst 33342 according to the method of Smith et al (Brit. Jnl. Cancer 1988, 57, 247-253). Five animals were used in control and treated groups. The fluorescent dye was dissolved in saline at 6.25 mg/ml and injected intravenously at 10 mg/kg 24 hours after intraperitoneal drug treatment. One minute later, animals were killed and tumours excised and frozen; 10 μm sections were cut at 3 different levels and observed under UV illumination using an Olympus microscope equipped with epifluorescence. Blood vessels were identified by their fluorescent outlines and vascular volume was quantified using a point scoring system based on that described by Chalkley, (Jnl. Natl. Cancer Inst., 1943, 4, 47-53). All estimates were based on counting a minimum of 100 fields from sections cut at the 3 different levels.

The ability of the compounds to bind to preparations of mammalian tubulin can be evaluated by a number of methods available in the literature, for example by following temperature initiated tubulin polymerisation by turbidity in the absence and presence of the compound (for example O. Boye et al Med. Chem. Res., 1991, 1, 142-150).

The activity of N-[3-amino-9,10,11-trimethoxy-6,7-dihydro-5H-dibenzo[a,c]cyclohepten-5-yl]acetamide, (V. Fernholz Justus Liebigs Ann., 1950, 568, 63-72), against tumour vasculature was measured by the fluorescent dye method described above. This compound decreased perfused vascular volume by 88% relative to control when dosed at 50 mg/kg intraperitoneally. The IC₅₀ of this compound in a tubulin polymerisation assay was 58 micromolar (O. Boye et al Med. Chem. Res., 1991, 1, 142-150).

(c) HUVEC Detachment Assay

This assay examined the effects of compounds on the adherence of HUVECs to tissue culture plasticware.

HUVECs were plated in 0.2% gelatin-coated 12 well tissue culture plates at a concentration of 3×10⁴ cells per well in 1 ml TCS medium. After 24 hours, when the cells were at ˜30% confluency, the cells were dosed with compound for 40 minutes at 37° C., 5% CO₂. After this incubation the medium containing drug was pipetted off, and the cells were then gently washed in 2 mls of HBSS (Hanks' Balanced Salt Solution purchased from Life Technologies Ltd, Paisley UK; Catalogue # 24020-083) to remove any detached cells. The washing solution was then removed, and the adherent cells remaining were trypsinised using 300 μl of 1× Trypsin-EDTA solution (Life Technologies Ltd, Paisley, UK; Catalogue # 43500-019) at ambient temperature for 2 minutes. The trypsinised cells were then made up to 1 ml with TCS Biologicals medium, then centrifuged at 2000 rpm for 2 minutes. The cell pellet was then resuspended in a volume of 50 μl of TCS Biologicals medium. Total cell counts were obtained by counting the cells on a haemocytometer. The amount of cell detachment was calculated by comparing the number of cells remaining attached following treatment with the number in undosed control wells.

(d) Hras5 Necrosis Model

NIH 3T3 fibroblasts transfected with Harvey ras, clone 5, (Hras5 cells) were kept in continual passage in Dulbecco's modifed Eagles medium (DMEM) containing 10% foetal bovine serum (FBS) and 1% glutamine, at 37° C. in a humidified incubator gassed with 7.5% carbon dioxide and 92.5% oxygen. Cells were implanted subcutaneously into the left flank of male nude mice (8-10 weeks of age) at an inoculum of 2×10⁵ cells/mouse. Tumours were measured using calipers and randomised into groups of 2-4 mice between days 9-14 after implant. Mice were dosed with compounds, either intravenously or intraperitoneally, once on day of randomisation and culled 24 hours after dosing. Compounds were dissolved in 20% hydroxypropyl beta cyclodextrin in physiological saline at pH 7 and dosed in a volume of 0.1 ml per 10 g body weight. Tumours were excised, weighed and placed in buffered formalin. Area of necrosis in individual tumours was assessed from a haematoxylin/eosin stained-slide by a pathologist and scored from 0, meaning no significant change, to 10, meaning 91-100% necrosis. The activity of examples 5 and 7 (described hereinafter) against tumour vasculature was measured by the fluorescent dye method described hereinabove. Example 1 scored 6.6 at 25 mg/kg.

According to a further aspect of the invention there is provided a pharmaceutical composition which comprises a compound of the formula I as defined hereinbefore or a pharmaceutically acceptable salt, solvate or pro-drug thereof, in association with a pharmaceutically acceptable excipient or carrier.

The composition may be in a form suitable for oral administration, for example as a tablet or capsule, for nasal administration or administration by inhalation, for example as a powder or solution, for parenteral injection (including intravenous, subcutaneous, intramuscular, intravascular or infusion) for example as a sterile solution, suspension or emulsion, for topical administration for example as an ointment or cream or for rectal administration for example as a suppository. In general the above compositions may be prepared in a conventional manner using conventional excipients.

The compositions of the present invention are advantageously presented in unit dosage form. The compound will normally be administered to a warm-blooded animal at a unit dose within the range 5-5000 mg per square metre body area of the animal, i.e. approximately 0.1-100 mg/kg. A unit dose in the range, for example, 1-100 mg/kg, preferably 1-50 mg/kg is envisaged and this normally provides a therapeutically-effective dose. A unit dose form such as a tablet or capsule will usually contain, for example 1-250 mg of active ingredient.

As stated above the size of the dose required for the therapeutic or prophylactic treatment of a particular disease state will necessarily be varied depending on the host treated, the route of administration and the severity of the illness being treated. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

According to a further aspect of the present invention there is provided a compound of the formula I or a pharmaceutically acceptable salt, solvate or pro-drug thereof as defined hereinbefore for use in a method of treatment of the human or animal body by therapy.

A further feature of the present invention is a compound of formula I, or a pharmaceutically acceptable salt, solvate or pro-drug thereof, for use as a medicament, conveniently a compound of formula I, or a pharmaceutically acceptable salt, solvate or pro-drug thereof, for use as a medicament for producing a vascular damaging effect in a warm-blooded animal such as a human being.

Thus according to a further aspect of the invention there is provided the use of a compound of the formula I, or a pharmaceutically acceptable salt, solvate or pro-drug thereof in the manufacture of a medicament for use in the production of a vascular damaging effect in a warm-blooded animal such as a human being.

According to a further feature of the invention there is provided a method for producing a vascular damaging effect in a warm-blooded animal, such as a human being, in need of such treatment which comprises administering to said animal an effective amount of a compound of formula I or a pharmaceutically acceptable salt, solvate or pro-drug thereof as defined hereinbefore.

According to a further aspect of the present invention there is provided a compound of formula (I) or pharmaceutically-acceptable salt, solvate or pro-drug thereof, preferably in the form of a pharmaceutical composition, when dosed in divided doses (also known as split doses) produces a greater anti-tumour effect than when a single dose is given.

Anti-tumour effects of a method of treatment of the present invention include but are not limited to, inhibition of tumour growth, tumour growth delay, regression of tumour, shrinkage of tumour, increased time to re-growth of tumour on cessation of treatment, slowing of disease progression. It is expected that when a method of treatment of the present invention is administered to a warm-blooded animal such as a human, in need of treatment for cancer involving a solid tumour, said method of treatment will produce an effect, as measured by, for example, one or more of: the extent of the anti-tumour effect, the response rate, the time to disease progression and the survival rate.

According to a further aspect of the present invention there is provided a method for the production of a vascular damaging effect in a warm-blooded animal such as a human, which comprises administering to said animal in divided doses an effective amount of a compound of formula (I) or pharmaceutically-acceptable salt, solvate or pro-drug thereof, preferably in the form of a pharmaceutical composition.

According to a further aspect of the present invention there is provided a method for the treatment of a cancer involving a solid tumour in a warm-blooded animal such as a human, which comprises administering to said animal in divided doses an effective amount of a compound of formula (I) or pharmaceutically-acceptable salt or solvate thereof, preferably in the form of a pharmaceutical composition.

According to a further aspect of the present invention there is provided a medicament comprising two or more fractions of doses of a compound of formula (I) or pharmaceutically-acceptable salt, solvate or pro-drug thereof, preferably in the form of a pharmaceutical composition, which together add up to a total daily dose, for administration in divided doses for use in a method of treatment of a human or animal body by therapy.

According to a further aspect of the present invention there is provided a kit comprising two or more fractions of doses of a compound of formula (I) or pharmaceutically-acceptable salt, solvate or pro-drug thereof, preferably in the form of a pharmaceutical composition, which together add up to a total daily dose, for administration in divided doses.

According to a further aspect of the present invention there is provided a kit comprising:
a) two or more fractions of doses of a compound of formula (I) or pharmaceutically-acceptable salt, solvate or pro-drug thereof, which together add up to a total daily dose, in unit dosage forms for administration in divided doses; and
b) container means for containing said dosage forms.

According to a further aspect of the present invention there is provided a kit comprising:
a) two or more fractions of doses of a compound of formula (I) or pharmaceutically-acceptable salt, solvate or pro-drug thereof, which together add up to a total daily dose, together with a pharmaceutically acceptable excipient or carrier, in unit dosage forms; and
b) container means for containing said dosage forms.

According to a further aspect of the present invention there is provided the use of compound of formula (I) or pharmaceutically-acceptable salt, solvate or pro-drug thereof in the manufacture of a medicament for administration in divided doses for use in the production of a vascular damaging effect in a warm-blooded animal such as a human.

According to a further aspect of the present invention there is provided the use of a compound of formula (I) or pharmaceutically-acceptable salt, solvate or pro-drug thereof in the manufacture of a medicament for administration in divided doses for use in the production of an anti-cancer effect in a warm-blooded animal such as a human.

According to a further aspect of the present invention there is provided the use of a compound of formula (I) or pharmaceutically-acceptable salt, solvate or pro-drug thereof in the manufacture of a medicament for administration in divided doses for use in the production of an anti-tumour effect in a warm-blooded animal such as a human.

Divided doses, also called split doses, means that the total dose to be administered to a warm-blooded animal, such as a human, in any one day period (for example one 24 hour period from midnight to midnight) is divided up into two or more fractions of the total dose and these fractions are administered with a time period between each fraction of about greater than 0 hours to about 10 hours, preferably about 1 hour to about 6 hours, more preferably about 2 hours to about 4 hours. The fractions of total dose may be about equal or unequal.

Preferably the total dose is divided into two parts which may be about equal or unequal.

The time intervals between doses may be for example selected from:

about 1 hour, about 1.5 hours, about 2 hours, about 2.5 hours, about 3 hours, about 3.5 hours, about 4 hours, about 4.5 hours, about 5 hours, about 5.5 hours and about 6 hours.

The time intervals between doses may be any number (including non-integers) of minutes between greater than 0 minutes and 600 minutes, preferably between 45 and 375 minutes inclusive. If more than two doses are administered the time intervals between each dose may be about equal or unequal.

Preferably two doses are given with a time interval in between them of greater than or equal to 1 hour and less than 6 hours.

More preferably two doses are given with a time interval in between them of greater than or equal to two hours and less than 5 hours.

Yet more preferably two doses are given with a time interval in between them of greater than or equal to two hours and less than or equal to 4 hours.

Particularly the total dose is divided into two parts which may be about equal or unequal with a time interval between doses of greater than or equal to about two hours and less than or equal to about 4 hours.

More particularly the total dose is divided into two parts which may be about equal with a time interval between doses of greater than or equal to about two hours and less than or equal to about 4 hours.

For the avoidance of doubt the term ‘about’ in the description of time periods means the time given plus or minus 15 minutes, thus for example about 1 hour means 45 to 75 minutes, about 1.5 hours means 75 to 105 minutes. Elsewhere the term ‘about’ has its usual dictionary meaning.

The antiangiogenic treatment defined hereinbefore may be applied as a sole therapy or may involve, in addition to a compound of the invention, one or more other substances and/or treatments. Such conjoint treatment may be achieved by way of the simultaneous, sequential or separate administration of the individual components of the treatment. In the field of medical oncology it is normal practice to use a combination of different forms of treatment to treat each patient with cancer. In medical oncology the other component(s) of such conjoint treatment in addition to the antiangiogenic treatment defined hereinbefore may be: surgery, radiotherapy or chemotherapy. Such chemotherapy may include the following categories of therapeutic agent: (i) other antiangiogenic agents that work by different mechanisms from those defined hereinbefore (for example linomide, inhibitors of integrin αvβ3 function, angiostatin, endostatin, razoxin, thalidomide) and including vascular endothelial growth factor (VEGF) receptor tyrosine kinase inhibitors (RTKIs) (for example those described in International Patent Applications Publication Nos. WO 97/22596, WO 97/30035, WO 97/32856 and WO 98/13354 the entire disclosure of which documents is incorporated herein by reference); (ii) cytostatic agents such as antioestrogens (for example tamoxifen, toremifene, raloxifene, droloxifene, iodoxyfene), progestogens (for example megestrol acetate), aromatase inhibitors (for example anastrozole, letrazole, vorazole, exemestane), antiprogestogens, antiandrogens (for example flutamide, nilutamide, bicalutamide, cyproterone acetate), LHRH agonists and antagonists (for example goserelin acetate, luprolide), inhibitors of testosterone 5α-dihydroreductase (for example finasteride), anti-invasion agents (for example metalloproteinase inhibitors like marimastat and inhibitors of urokinase plasminogen activator receptor function) and inhibitors of growth factor function, (such growth factors include for example epidermal growth factor (EGF), platelet derived growth factor and hepatocyte growth factor such inhibitors include growth factor antibodies, growth factor receptor antibodies, tyrosine kinase inhibitors and serine/threonine kinase inhibitors); (iii) biological response modifiers (for example interferon); (iv) antibodies (for example edrecolomab); and (v) antiproliferative/antineoplastic drugs and combinations thereof, as used in medical oncology, such as antimetabolites (for example antifolates like methotrexate, fluoropyrimidines like 5-fluorouracil, purine and adenosine analogues, cytosine arabinoside); antitumour antibiotics (for example anthracyclines like doxorubicin, daunomycin, epirubicin and idarubicin, mitomycin-C, dactinomycin, n-dthramycin); platinum derivatives (for example cisplatin, carboplatin); alkylating agents (for example nitrogen mustard, melphalan, chlorambucil, busulphan, cyclophospharide, ifosfamide, nitrosoureas, thiotepa); antimitotic agents (for example vinca alkaloids like vincristine and taxoids like taxol, taxotere); enzymes (for example asparaginase); thymidylate synthase inhibitors (for example raltitrexed); topoisomerase inhibitors (for example epipodophyllotoxins like etoposide and teniposide, amsacrine, topotecan, irinotecan).

As stated above the compounds defined in the present invention are of interest for their vascular damaging effects. Such compounds of the invention are expected to be useful in the prophylaxis and treatment of a wide range of disease states where inappropriate angiogenesis occurs including cancer, diabetes, psoriasis, rheumatoid arthritis, Kaposi's sarcoma, haemangioma, acute and chronic nephropathies, atheroma, arterial restenosis, autoimmune diseases, acute inflammation, endometriosis, dysfunctional uterine bleeding and ocular diseases with retinal vessel proliferation. In particular such compounds of the invention are expected to slow advantageously the growth of primary and recurrent solid tumours of, for example, the colon, breast, prostate, lungs and skin.

In addition to their use in therapeutic medicine, the compounds of formula I and their pharmaceutically acceptable salts, solvates and pro-drugs are also useful as pharmacological tools in the development and standardisation of in vitro and in vivo test systems for the evaluation of the effects of vascular damaging agents in laboratory animals such as cats, dogs, rabbits, monkeys, rats and mice, as part of the search for new therapeutic agents.

It is to be understood that where the term “ether” is used anywhere in this specification it refers to diethyl ether.

The invention will now be illustrated in the following non-limiting Examples in which, unless otherwise stated:

(i) evaporations were carried out by rotary evaporation in vacuo and work-up procedures were carried out after removal of residual solids such as drying agents by filtration;

(ii) operations were carried out at ambient temperature, that is in the range 18-25° C. and under an atmosphere of an inert gas such as argon or nitrogen;

(iii) yields are given for illustration only and are not necessarily the maximum attainable;

(iv) the structures of the end-products of the formula I were confirmed by nuclear (generally proton) magnetic resonance (NMR) and mass spectral techniques; proton magnetic resonance chemical shift values were measured on the delta scale and peak multiplicities are shown as follows: s, singlet; d, doublet; t, triplet; m, multiplet; br, broad; q, quartet, quin, quintet;

(v) intermediates were not generally fully characterised and purity was assessed by thin layer chromatography (TLC), high-performance liquid chromatography (HPLC), infra-red (IR) or NMR analysis;

Abbreviations

4-Dimethylaminopyridine DMAP
1-(3 -Dimethylaminopropyl)-3-ethylcarbodiimide hydrochloride EDCI
Dimethyl sulphoxide DMSO
Trifluoroacetic acid TFA

### EXAMPLE 1

### (5S)-5-Acetylamino-9,10,11-trimethoxy-6,7-dihydro-5H-dibenzo[a,c]cyclohepten-3-yl 5-(4-acetylpiperazin-1-yl)-5-oxopentanoate

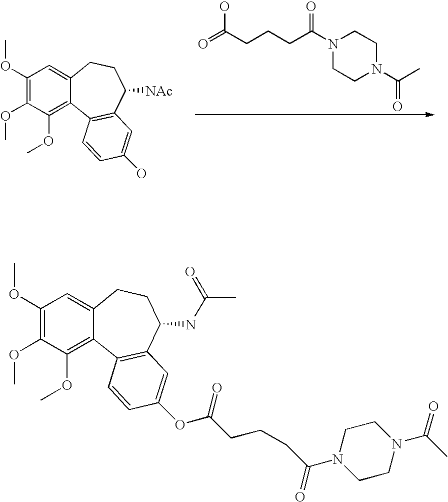

A solution of 5-(4-acetylpiperazin-1-yl)-5-oxopentanoic acid (0.308 g; 1.27 mmol), EDCI (0.244 g; 1.27 mmol), DMAP (0.036 g; 0.29 mmol) in dichloromethane (30 ml) was stirred under argon atmosphere for 30 minutes. N-acetyl colchinol [International Patent Application No. PCT/GB98/01977] (0.350 g; 0.98 mmol) was then added and the mixture was stirred overnight. After evaporation to dryness, the residue was purified by flash chromatography, eluting with dichloromethane/methanol (95/5) to give the title compound after evaporation of the appropriate fractions and trituration in ether/pentane.

Yield: 82%

¹H NMR (DMSO-d₆): 1.45-1.50 (m, 1H); 1.51-1.75 (m, 1H); 1.87 (s, 3H); 1.79-1.94 (m, 1H); 1.94-2.11 (m, 2H); 2.02 (s, 3H); 2.10-2.24 (m, 1H); 2.52-2.62 (m, 1H, signal partially obscured by DMSO peak); 2.74-2.85 (m, 1H); 2.88-2.98 (m, 1H); 3.14-3.24 (m, 1H); 3.28-3.33 (m, 1H); 3.51 (s, 3H); 3.78 (s, 3H); 3.78-3.89 (m, 1H); 3.85 (s, 3H); 4.24-4.33 (m, 1H); 4.49-4.59 (m, 1H); 6.80 (s, 1H); 7.07 (s, 1H); 7.09 (dd, 1H); 7.35 (d, 1H); 3.39 (d, 1H).

MS-ESI: 582 [MH]⁺

| | | | | |
|---|---|---|---|---|
| Elemental analysis | Found | C 62.54 | H 6.92 | N 6.93 |
| C₃₁H₃₉N₃O₈, 0.8H₂O | Requires | C 62.47 | H 6.87 | N 7.05 |

The starting material as follows:

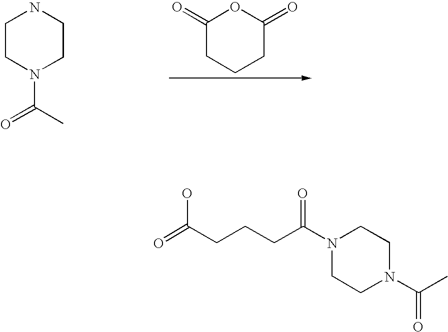

A solution of glutaric anhydride (1.6 g; 14 mmol) and N-acetylpiperazine (1.5 g; 12 mmol) in dichloromethane (20 ml) was stirred overnight. The resulting precipitate was filtered, washed with ether and dried to give 5-(4-acetylpiperazin-1-yl)-5-oxopentanoic acid as a white solid.

Yield: 83%

¹H NMR (CDCl₃): 1.98 (m, 2H); 2.13 (s, 3H); 2.46 (m, 4H); 3.47 (m, 4H); 3.64 (m, 4H).

### EXAMPLE 2

### (5S)-5-Acetylamino-9,10,11-trimethoxy-6,7-dihydro-5H-dibenzo[a,c}cyclohepten-3-yl 4-(4-acetylpiperazin-1-yl)-4-oxobutanoate

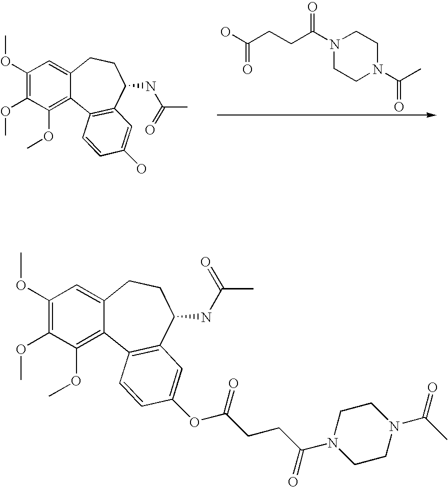

The compound was prepared using a similar method to that described in Example 1, but using 4-(4-acetylpiperazin-1-yl)-4-oxobutanoic acid in place of 5-(4-acetylpiperazin-1-yl)-5-oxopentanoic acid.

Yield: 67%

¹H NMR (DMSO-d₆): 1.81-1.93 (m, 1H); 1.86 (s, 3H); 1.98-2.10 (m, 1H); 2.02 (d, 3H); 2.11-2.23 (m, 1H); 2.52-2.59 (m, 1H, signal partially obscured by DMSO peak); 2.71-2.85 (m, 4H); 3.27-3.54 (m, 8H); 3.51 (s, 3H); 3.78 (s, 3H); 3.84 (s, 3H); 4.47-4.58 (m, 1H); 6.80 (s, 1H); 7.04 (dd, 1H); 7.06 (d, 1H); 7.34 (d, 1H); 8.41 (d, 1H).

MS-ESI: 568 [MH]⁺

The starting material was prepared as follows:

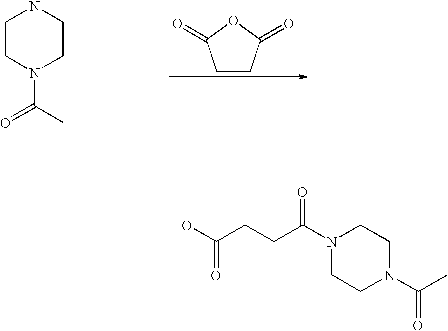

A solution of succinic anhydride (1.72 g; 17 mmol) and N-acetyl piperazine (2 g; 15.6 mmol) in dichloromethane (40 ml) was stirred overnight. After evaporation to dryness, the residue was triturated in ether/pentane to give 4-(4-acetylpiperazin-1-yl)-4-oxobutanoic acid as a solid.

Yield: 95%

¹H NMR (CDCl₃): 2.13 (s, 3H); 2.70 (m, 4H); 3.48 (m, 4H); 3.66 (m, 4H).

### EXAMPLE 3

### (5S)-5-Acetylamino-9,10,11-trimethoxy-6,7-dihydro-5H-dibenzo[a,c]cyclohepten-3-yl 3-(4-acetylpiperazin-1-ylmethyl)benzoate

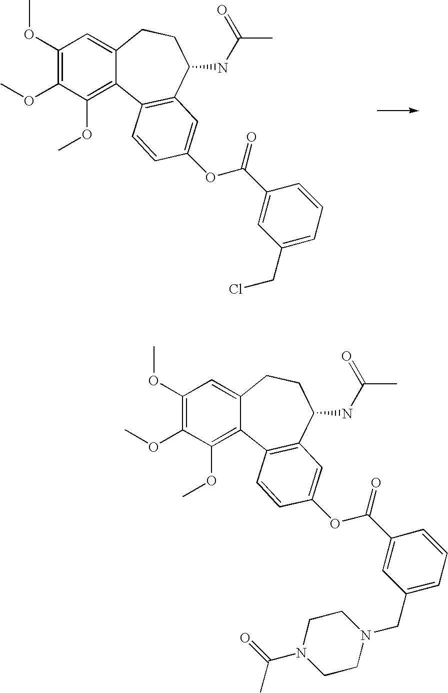

A solution of (5S)-5-acetylamino-9,10,11-trimethoxy-6,7-dihydro-5H-dibenzo[a,c]cyclohepten-3-yl 3-chloromethylbenzoate (0.408 g; 0.8 mmol), N-acetylpiperazine (0.144 g; 1.12 mmol) and sodium iodide (0.06 g; 0.4 mmol) in acetonitrile (6 ml) was stirred under argon atmosphere at room temperature overnight. After evaporation to dryness, the mixture was purified by flash chromatography eluting with dichloromethane/ethanol (92/8) to give the title compound as a white solid.

Yield: 69%

¹H NMR (DMSO-d₆): 1.81-1.97 (m, 1H); 1.87 (s, 3H); 1.99 (s, 3H); 2.04-2.28 (m, 2H); 2.28-2.38 (m, 2H); 2.39-2.48 (m, 2H); 2.48-2.55 (m, 1H, signal partially obscured by DMSO peak); 3.42-3.51 (m, 4H); 3.56 (s, 3H); 3.64 (s, 2H); 3.81 (s, 3H); 3.87 (s, 3H); 4.54-4.67 (m, 1H); 6.84 (s, 1H); 7.25 (s, 1H); 7.26 (dd, 1H); 7.43 (d, 1H); 7.52 (dd, 1H); 7.72 (d, 1H); 8.10 (d, 1H); 8.12 (s, 1H); 8.40 (d, 1H).

MS-ESI: 602 [MH]⁺

| | | | | |
|---|---|---|---|---|
| Elemental analysis | Found | C 67.00 | H 6.76 | N 6.81 |
| C₃₄H₃₉N₃O₇, 0.3H₂O | Requires | C 67.27 | H 6.57 | N 6.92 |

### EXAMPLE 4

### (5S)-5-Acetylamino-9,10,11-trimethoxy-6,7-dihydro-5H-dibenzo[a,c]cyclohepten-3-yl 4-[3-(4-methylpiperazin-1-yl)propionylamino]benzoate

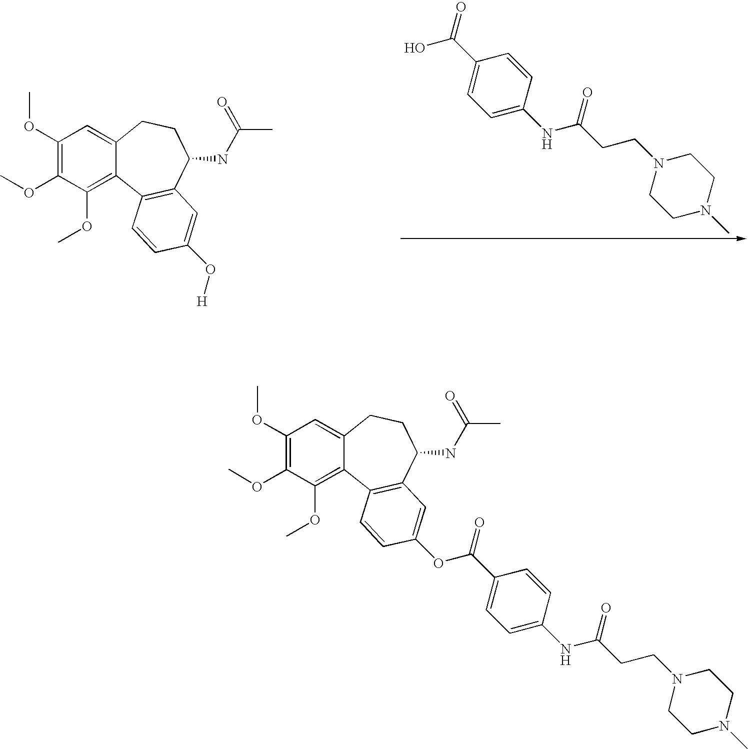

The title compound was prepared using a similar method to that in Example 1 but replacing 5-(4-acetylpiperazin-1-yl)-5-oxopentanoic acid by 4-{3-(4-methylpiperazin-1-yl)propionylamino]benzoic acid.

Yield: 55%

¹H NMR (DMSO-d₆): 1.78-2.75 (m, 14H); 1.88 (s, 3H); 2.17 (s, 3H); 2.64 (t, 2H); 3.56 (s, 3H); 3.81 (s, 3H); 3.83 (s, 3H); 4.51-4.65 (m, 1H); 6.82 (s, 1H); 7.19-7.24 (m, 2H); 7.40 (d, 1H); 7.82 (d, 2H); 8.12 (d, 2H); 8.39 (d, 1H); 10.53 (s, 1H).

MS-ESI: 631 [MH]⁺

The starting material was prepared as follows:

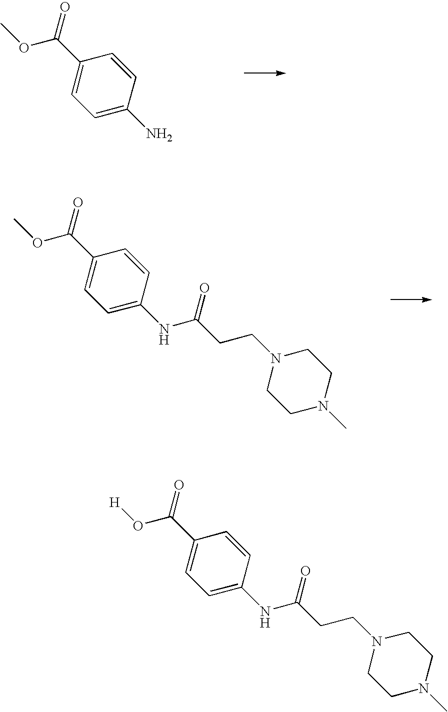

A solution of methyl 4-aminobenzoate (0.76 g; 5 mmol), EDCI (1.25 g; 6.5 mmol), DMAP (0.13 g; 1 mmol), 3-(4-methylpiperazin-1-yl)propanoic acid (1.49 g; 7.5 mmol) and triethylamine (1.05 ml; 7.5 mmol) in dichloromethane (20 ml) was stirred under argon atmosphere for 2 days. The mixture was extracted with ethyl acetate, evaporated and purified by flash chromatography eluting with dichloromethane/methanol to give methyl 4-[3-(4-methylpiperazin-1-yl)propionylamino]benzoate.

Yield: 46%

¹H NMR (DMSO-d₆): 2.14 (s, 3H); 2.07-2.70 (m, 10H); 2.62 (t, 2H); 3.82 (s, 3H); 7.71 (d, 2H); 7.91 (d, 2H); 10.41 (s, 1H).

MS-ESI: 306 [MH]⁺

A solution of methyl 4-[3-(4-methylpiperazin-1-yl) propionylamino]benzoate (0.69 g; 2.26 mmol) in methanol (10 ml) was treated with 2N sodium hydroxide solution (1.25 ml; 2.48 mmol) at 60° C. for 6 hours. After evaporation to dryness, the residue was triturated in acetone. The insoluble material was taken-up in water and the pH adjusted to 6.5 with 2N HCl. After evaporation, the residue was triturated in acetone to give 4-[3-(4-methylpiperazin-1-yl)propionylamino]benzoic acid as a solid.

Yield: 98%.

¹H NMR (DMSO-d₆): 2.17 (s, 3H); 2.10-2.70 (m, 10H); 2.64 (t, 2H); 7.69 (d, 2H); 7.88 (d, 2H).

### EXAMPLE 5

### (5S)-5-Acetylamino-9,10,11-trimethoxy-6,7-dihydro-5H-dibenzo[a,c]cyclohepten-3-yl 3-(4-carbamoylpiperazin-1-ylmethyl)benzoate

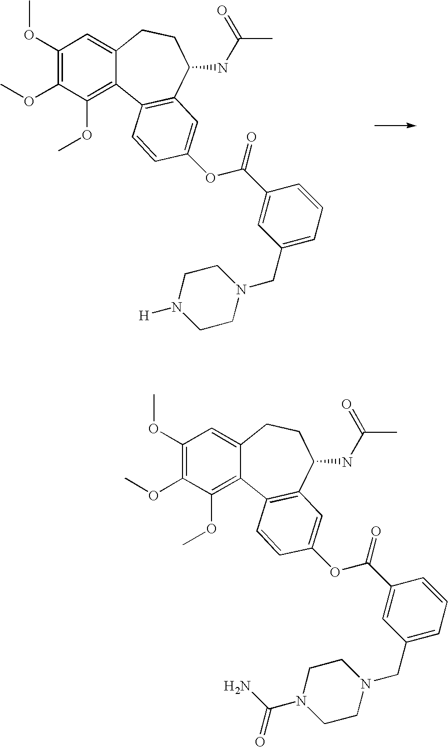

A solution (5S)-5-acetylamino-9,10,11-trimethoxy-6,7-dihydro-5H-dibenzo[a,c]cyclohepten-3-yl 3-(piperazin-1-ylmethyl)benzoate (0.2 g; 0.357 mmol) and trimethylsilyl isocyanate (0.290 ml; 2.14 mmol) in dichloromethane (3 ml) was stirred at ambient temperature overnight. After evaporation to dryness, the residue was purified by flash chromatography, eluting with dichloromethane/ethanol (85/15) to give the title compound.

Yield: 87%

¹H NMR (DMSO-d₆: 1.80-1.96 (m, 1H); 1.88 (s, 3H); 2.00-2.28 (m, 2H); 2.35 (m, 4H); 2.52-2.59 (m, 1H, signal partially obscured by DMSO peak); 3.30 (m, 4H); 3.56 (s, 3H); 3.63 (s, 2H); 3.81 (s, 3H); 3.87 (s, 3H); 4.55-4.54 (m, 1H); 5.94 (s, 2H); 6.82 (s, 1H); 7.23 (s, 1H); 7.25 (dd, 1H); 7.41 (d, 1H); 7.59 (t, 1H); 7.70 (d, 1H); 8.07 (d, 1H); 8.10 (s, 1H); 8.39 (d, 1H).

MS-ESI: 603 [MH]⁺

The starting material was prepared as follows

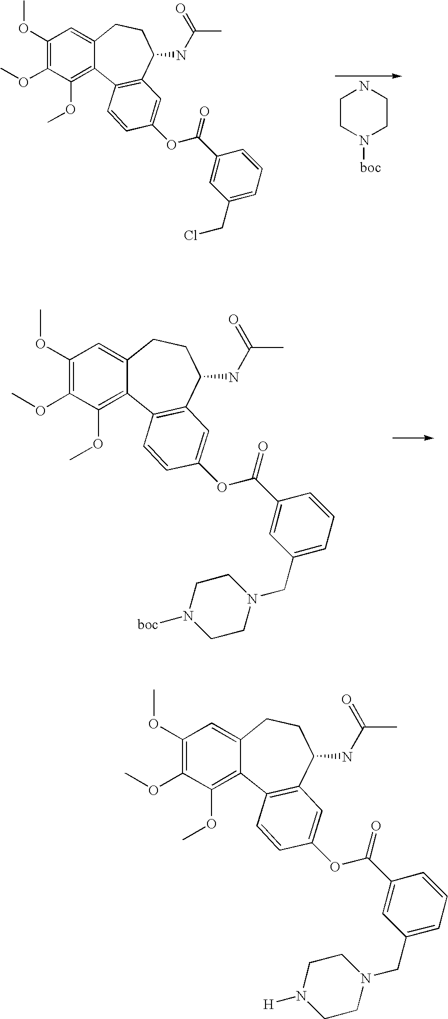

A solution of (5S)-5-acetylamino-9,10,11-trimethoxy-6,7-dihydro-5H-dibenzo[a,c]cyclohepten-3-yl 3-chloromethylbenzoate (0.714 g; 1.4 mmol), N-tert-butoxycarbonyl piperazine (0.417 g; 2.24 mmol) and sodium iodide (0.21 g; 1.4 mmol) in dichloromethane (20 ml) was stirred at 45° C., under argon atmosphere for 24 hours. After evaporation to dryness, the residue was purified by flash chromatography eluting with dichloromethane/ethanol (95/5) to give (5S)-5-acetylamino-9,10,11-trimethoxy-6,7-dihydro-5H-dibenzo[a,c]cyclohepten-3-yl 3-(4-tert-butoxycarbonylpiperazin-1-ylmethyl)benzoate.

Yield: 55%

¹H NMR (DMSO-d₆): 1.39 (s, 9H); 1.81-1.96 (m, 1H); 1.86 (s, 3H); 2.02-2.26 (m, 2H); 2.36 (t, 4H); 2.53-2.60 (m, 1H, signal partially obscured by DMSO peak); 3.34 (m, 8H); 3.54 (s, 3H); 3.61 (s, 2H); 3.80 (s, 3H); 3.85 (s, 3H); 4.53-4.64 (m, 1H); 6.82 (s, 1H); 7.23 (s, 1H); 7.24 (dd, 1H); 7.41 (d, 1H); 7.59 (t, 1H); 7.70 (d, 1H); 8.07 (d, 1H); 8.10 (s, 1H); 8.38 (d, 1H).

MS-ESI: 660 [MH]⁺

A solution of (5S)-5-acetylamino-9,10,11-trimethoxy-6,7-dihydro-5H-dibenzo[a,c]cyclohepten-3-yl 3-(4-tert-butoxycarbonylpiperazin-1-ylmethyl)benzoate (0.711 g; 1.07 mmol) in dichloromethane (15 ml) was treated with 2.5N HCl/ether (3.5 ml) at ambient temperature for 1 hour. After evaporation, the residue was taken-up in water, the pH was adjusted to 5 with 2N sodium hydroxide solution and the solution purified on reverse phase silica, eluting with a gradient of 40-50% methanol/ammonium carbonate buffer (2 g/l pH 7) to give (5S)-5-acetylamino-9,10,11-trimethoxy-6,7-dihydro-5H-dibenzo[a,c]cyclohepten-3-yl 3-(piperazin-1-ylmethyl)benzoate.

Yield: 55%

¹H NMR (DMSO-d₆): 1.82 -1.97 (m, 1H); 1.88 (s, 3H); 2.02-2.26 (m, 2H); 2.33 (bs, 4H); 2.51-2.60 (m, 1H); 2.71 (m, 4H); 3.56 (s, 3H); 3.82 (s, 3H); 3.87 (s, 3H); 4.45-4.64 (m, 1H); 6.84 (s, 1H); 7.24 (s, 1H); 7.25 (dd, 1H); 7.41 (d, 1H); 7.58 (t, 1H); 7.69 (d, 1H); 8.05 (d, 1H); 8.08 (s, 1H); 8.38 (d, 1H).

MS-ESI: 560 [MH]⁺

### EXAMPLE 6

(5S)-5-Acetylamino-9,10,11-trimethoxy-6,7-dihydro-5H-dibenzo[a,c]cyclohepten-3-yl N-acetylpiperidin-1-ylcarboxylate

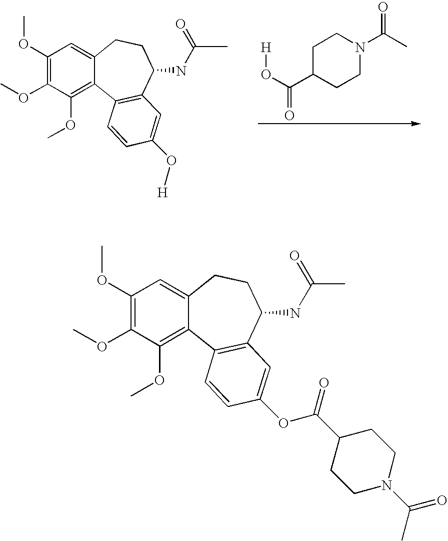

The title compound was prepared using a similar method to that described in Example 1, but 4-acetylpiperidin-1-ylcarboxylic acid was used in place of 5-(4-acetylpiperazin-1-yl)-5-oxopentanoic acid:

Yield: 79%

¹H NMR (DMSO-d₆): 1.45-1.60 (m, 1H); 1.61-1.75 (m, 1H); 1.88-1.93 (m, 2H); 1.87 (s, 3H); 1.94-2.09 (m, 2H); 2.02 (s, 3H); 2.10-2.24 (m, 1H); 2.53-2.60 (m, 1H, signal partially obscured by DMSO peak); 2.75-2.85 (m, 1H); 2.88-2.98 (m, 1H); 3.14-3.25 (m, 1H); 3.26 (bs, 1H); 3.50 (s, 3H); 3.78 (s, 3H); 3.81 (bs, 1H); 3.84 (s, 3H); 4.23-4.33 (m, 1H); 4.49-4.59 (m, 1H); 6.80 (s, 1H); 7.06 (s, 1H); 7.08 (dd, 1H); 7.35 (d, 1H); 8.39 (d, 1H).

MS-ESI: 511 [MH]⁺

| | | | | |
|---|---|---|---|---|
| Elemental analysis | Found | C 64.65 | H 6.85 | N 5.43 |
| C₂₈H₃₃N₂O₇, 0.5H₂O | Requires | C 64.85 | H 6.61 | N 5.40 |

### EXAMPLE 7

### (5S)-5-Acetylamino-9,10,11-trimethoxy-6,7-dihydro-5H-dibenzo[a,c]cyclohepten-3-yl 3-[N,N-di-(2-hydroxyethyl)carbamoyl]propanoate

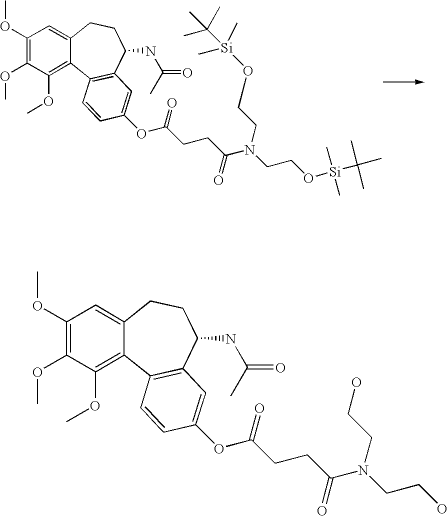

A 2.4 N solution of sulphuric acid in methanol (4 ml) was added at 3° C. under argon atmosphere, to a solution of (5S)-5-acetylamino-9,10,11-trimethoxy-6,7-dihydro-5H-dibenzo[a,c]cyclohepten-3-yl 3-{N—N-di-[2-(tert-butyldimethylsilyloxy)ethyl]carbamoyl}propanoate (0.7 g; 0.906 mmol) in methanol (15 ml). After stirring at 3° C. for 45 mins, a mixture of ethyl acetate (100 ml) and saturated aqueous sodium hydrogen carbonate solution (75 ml) was added. The organic phase was washed with water, dried and was purified by flash chromatography eluting dichloromethane/methanol (92/8) to give the title compound.

Yield: 66%

¹H NMR (DMSO-d₆): 1.81-1.96 (m, 1H); 1.89 (s, 3H); 1.98-2.56 (m, 2H); 2.55-2.65 (m, 1H, signal partially obscured by DMSO peak); 2.80 (bs, 4H); 4.37-4.63 (m, 8H); 3.53 (s, 3H); 3.80 (s, 3H); 3.86 (s, 3H); 4.49-4.60 (m, 1H); 4.69 (t, 1H); 4.88 (t, 1H); 6.80 (s, 1H); 7.02-7.07 (m, 2H); 7.34 (d, 1H). 8.40 (d, 1H).

MS-ESI: 545 [MH]⁺

| | | | | |
|---|---|---|---|---|
| Elemental analysis | Found | C 59.27 | H 6.62 | N 4.89 |
| C₂₈H₃₆N₂O₉, 1.2H₂O | Requires | C 59.40 | H 6.84 | N 4.95 |

The starting material was prepared as follows:

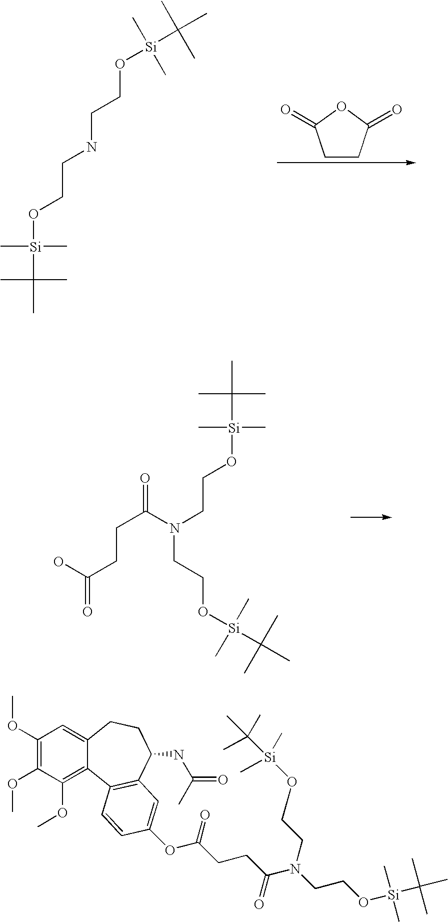

Succinic anhydride (2.64 g; 0.026 mol) was added under argon atmosphere to a solution of N—N-di-[2-(tert-butyldimethylsilyloxy)ethyl]amino [Synthesis (19997), 6, 643-648] (8 g; 0.024 mol) in dichloromethane. The mixture was stirred at ambient temperature overnight. After filtration of the insoluble, the filtrate was evaporated and dried to give 3-{N,N-di[2-(tert-butyldimethylsilyloxy)ethyl]carbamoyl}propanoic acid.

Yield: 96%

¹H NMR (CDCl₃): 0.04 (s, 6H); 0.05 (s, 6H); 0.87 (s, 9H); 0.88 (s, 9H); 2.62-2.69 (m, 2H); 2.83-2.90 (m, 2H); 3.52 (t, 2H); 3.58 (t, 2H); 3.75 (t, 2H); 3.78 (t, 2H).

A solution of 3-{N,N-di[2-(tert-butyldimethylsilyloxy)ethyl]carbamoyl}propanoic acid (0.552 g; 1.27 mmol), EDCI (0.244 g; 1.27 mmol), DMAP (0.036 g; 0.294 mmol) in dichloromethane (30 ml) was stirred under argon atmosphere for 30 mins. N-acetyl colchinol (0.35 g; 0.98 mmol) was added and the mixture was stirred at ambient temperature overnight. After evaporation to dryness, the residue was purified by flash chromatography, eluting with ethyl acetate/petroleum ether (70/30) to give 5(S)-5-acetylamino-9,10,1 1-trimethoxy-6,7-dihydro-5H-dibenzo[a,c]cyclohepten-3-yl 3 {N—N-di[2-(tert-butyl-dimethylsilyloxy]ethyl}carbamoyl propanoate.

Yield : 74%

¹H NMR (DMSO-d₆): 0.03 (s, 6H); 0.04 (s, 6H); 0.86 (s, 9H); 0.87 (s, 9H); 1.83-1.94 (m, 1H); 1.80 (s, 3H); 2.00-2.11 (m, 1H); 2.12-2.24 (m, 1H); 2.53-2.60 (m 1H, signal partially obscured by DMSO peak); 2.72-2.78 (m, 2H); 2.78-2.86 (m, 2H); 3.42 (t, 2H); 3.52 (s, 3H); 3.53 (t, 2H); 3.57 (t, 2H); 3.75 (t, 2H); 3.80 (s, 3H); 3.85 (s, 3H); 4.50-4.60 (m, 1H); 6.81 (s, 1H); 7.03 (dd, 1H); 7.07 (d, 1H); 7.34 (d, 1H); 8.41 (d, 1H).

### EXAMPLE 8

### (5S)-5-Acetylanamino-9,10,11-trimethoxy-6,7-dihydro-5H-dibenzor[a,c]cyclohepten-3-yl 4-[N,N-di(2-hydroxyethyl)carbamoyl]butanoate

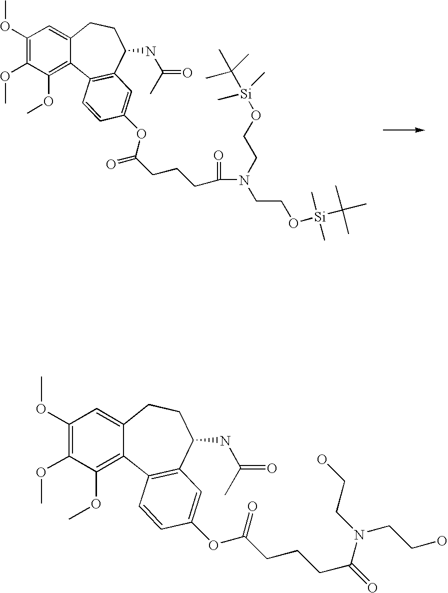

The title compound was prepared using a similar method to that described in Example 7, but using (5S)-5-acetylamino-9,10,11-trimethoxy-6,7-dihydro-5H-dibenzo[a,c}cyclohepten-3-yl 4-N,N-di[2-(tert-butyldimethylsilyloxy)ethyl]carbamoyl}butanoate in place of (5S)-5-acetylamino-9,10,11-trimethoxy-6,7-dihydro-5H-dibenzo[a,c]cyclohepten-3-yl 3 {N,N-di[2-(tert-butyl-dimethylsilyloxy)ethyl]carbamoyl}propanoate.

Yield: 37%

¹H NMR (DMSO-d₆): 1.83-1.92 (m, 2H); 1.87 (s, 3H); 1.98-2.10 (m, 1H); 2.11-2.22 (m, 1H); 2.48 (t, 2H, signal partially obscured by DMSO peak); 2.52-2.56 (m, 1H, signal partially obscured by DMSO peak); 2.63 (t, 2H); 3.35 (t, 2H); 3.41 (t, 2H); 3.44-3.50 (m, 2H); 3.50-3.57 (m, 2H); 3.51 (s, 3H); 3.78 (s, 3H); 3.84 (s, 3H); 4.50-4.60 (m, 1H); 4.67 (t, 1H); 4.85 (t, 1H); 6.80 (s, 1H); 7.07 (s, 1H); 7.09 (dd, 1H); 7.34 (d, 1H). 8.38 (d, 1H).

MS-ESI: 581 [MNa]⁺

| | | | | |
|---|---|---|---|---|
| Elemental analysis | Found | C 61.22 | H 7.21 | N 4.89 |
| C₂₉H₃₈N₂O₉, 0.5H₂O | Required | C 61.36 | H 6.93 | N 4.94 |

The starting material was prepared as follows:

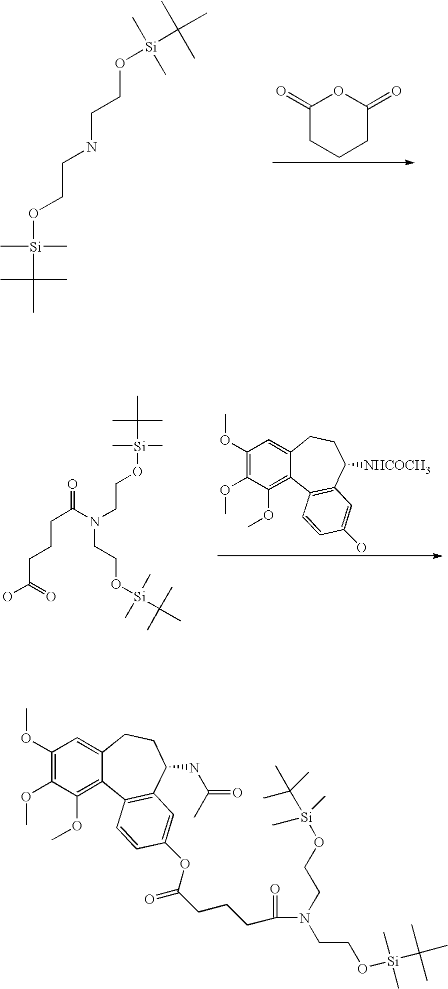

A solution of glutaric anhydride (0.753 g; 6.6 mmol) and N,N-di{2-(tert-butyldimethylsilyloxy)ethyl]amino (2 g; 6.6 mmol) in dichloromethane (40 ml) was stirred overnight. After evaporation to dryness the residue was triturated in pentane to give a solid which was filtered. The filtrate was evaporated to give 4-{N,N-di[2-(tert-butyldimethylsilyloxy)ethyl]carbamoyl}butanoic acid as an oil.

Yield: 95%

¹H NMR (DMSO-d₆): 0.03 (s, 6H); 0.04 (s, 6H); 0.86 (s, 9H); 0.87 (s, 9H); 1.70 (m, 2H); 2.23 (t, 2H); 2.38 (t, 2H); 3.38 (t, 2H); 3.46 (t, 2H); 3.65 (t, 2H); 3.70 (t, 2H).

4-{N,N-di[2-(tert-Butyldimethylsilyloxy)ethyl]carbamoyl}butanoic acid was reacted with N-acetyl colchinol using the similar conditions to that in Example 7, but using 4-{N,N-di[2-(tert-butyldimethylsilyloxy)ethyl]carbamoyl}butanoic acid in place of 3-{(N,N-di[2-(tert-butyldimethylsilyloxy)ethyl]carbamoyl}propanoic acid.

Yield: 92%

¹H NMR (DMSO-d₆): 0.04 (s, 12H); 0.86 (s, 9H); 0.87 (s, 9H); 1.83-1.94 (m, 2H); 1.88 (s, 3H); 2.00-2.11 (m, 1H); 2.12-2.24 (m, 1H); 2.48 (t, 2H, signal partially obscured by DMSO peak); 2.51-2.54 (m, 1H, signal partially obscured by DMSO peak); 2.65 (t, 2H); 3.41 (t, 2H); 3.50 (t, 2H); 3.53 (s, 3H); 3.64-3.76 (m, 2H); 3.80 (s, 3H); 3.86 (s, 3H); 4.51-4.61 (m, 1H); 6.81 (s, 1H); 7.08 (dd, 1H); 7.09 (d, 1H); 7.36 (d, 1H); 7.99 (s, 1H); 8.40 (d, 1H).

1. A compound of the formula I: 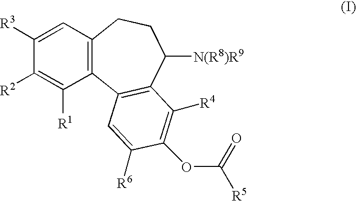
wherein:
R¹, R² and R³ are each independently hydroxy, phosphoryloxy (—OPO₃H₂), C₁₋₄alkoxy or an in vivo hydrolysable ester of hydroxy; with the proviso that at least two of R¹, R² and R³ are C₁₋₄alkoxy;
R⁴ and R⁶ are each independently selected from: hydrogen, nitro, amino, N—C₁₋₄alkylamino, N,N-di(C₁₋₄alkyl)amino, hydroxy, fluoro, C₁₋₄alkoxy and C₁₋₄alkyl;
R⁵ is selected from one of the following groups:
1) of the formula -A-X¹—Y¹—B, wherein:
A is C₁₋₄alkylene or —(CH₂)ₚ-Q- (wherein p is 0, 1 or 2 and Q is phenylene or thienylene);
X¹ is —O—, —CO—, —C(O)O—, —CON(R¹⁰)—, —N(R¹⁰)—, —N(R¹⁰)CO—, N(R¹⁰)C(O)O—, —N(R¹⁰)SO₂—, —SO₂N(R¹⁰)— or OC(O)N(R¹⁰)— (wherein R¹⁰ is hydrogen, C₁₋₃alkyl, hydroxyC₂₋₃alkyl, aminoC₂₋₃alkyl or C₁₋₃alkoxyC₂₋₃alkyl);
Y¹ is C₁₋₃alkylene;
B is carboxy, sulpho, phosphoryloxy, hydroxy, amino, N—(C₁₋₄alkyl)amino, N,N-di(C₁₋₃alkyl)amino, —R¹² or —NHC(R¹³)COOH; (wherein R¹² is a 5-6-membered saturated heterocyclic group (linked via carbon or nitrogen) containing 1 or 2 ring heteroatoms, selected independently from O, S and N, which heterocyclic group is optionally substituted by 1 or 2 substituents selected from: oxo, hydroxy, halogeno, C₁₋₄alkyl, C₂₋₄alkanoyl, carbamoyl, N—C₁₋₄alkylcarbamoyl, N,N-di-(C₁₋₄alkyl)carbamoyl, hydroxyC₁₋₄alkyl, C₁₋₄alkoxy, cyanoC₁₋₃alkyl, carbamoylC₁₋₃alkyl, carboxyC₁₋₄alkyl, aminoC₁₋₄alkyl, N,N-di(C₁₋₄alkyl)amino-C₁₋₄alkyl, C₁₋₄alkoxyC₁₋₄alkyl, C₁₋₄alkylsulphonylC₁₋₄alkyl and R¹⁴ (wherein R¹⁴ is a 5-6-membered saturated heterocyclic group (linked via carbon or nitrogen) containing 1 or 2 ring heteroatoms, selected independently from O, S and N, which heterocyclic group is optionally substituted by 1 or 2 substituents selected from:
oxo, hydroxy, halogeno, C₁₋₄alkyl, hydroxyC₁₋₄alkyl, C₁₋₄alkoxy, C₁₋₄alkoxy-C₁₋₄alkyl and C₁₋₄alkylsulphonylC₁₋₄alkyl));
R¹³ is an amino acid side chain;
2) of the formula: 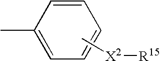
wherein:
the phenyl ring is substituted by —X²—R¹⁵ in the 3- or 4-position;
X² is —CO— or of the formula —(CH₂)ᵣ— (wherein r is 0, 1, 2 or 3) and
R¹⁵ is a 5-6 membered saturated heterocyclic group (linked via a ring carbon or nitrogen atom) containing 1 or 2 ring heteroatoms selected from O, S and N, which heterocyclic group is optionally substituted by 1 or 2 substituents selected from oxo, hydroxy, halogeno, C₁₋₄alkyl, C₂₋₄alkanoyl, carbamoyl, N—C₁₋₄alkylcarbamoyl, N,N-di-(C₁₋₄alkyl)carbamoyl, hydroxyC₁₋₄alkyl, C₁₋₄alkoxy, cyanoC₁₋₃alkyl, carbamoylC₁₋₃alkyl, carboxyC₁₋₄alkyl, C₁₋₄aminoalkyl, N,N-di(C₁₋₄alkyl)aminoC₁₋₄alkyl, C₁₋₄alkoxyC₁₋₄alkyl, C₁₋₄alkylsulphonylC₁₋₄alkyl and R¹⁴ (wherein R¹⁴ is as hereinabove defined); provided that the heterocyclic group (R¹⁵) is substituted by at least one substituent selected from C₂₋₄alkanoyl, carbamoyl, N—C₁₋₄alkylcarbamoyl and N,N-di(C₁₋₄alkyl)carbamoyl;
3) —(CH₂)ₐ—Y—(CH₂)_{b}—R¹⁵
(wherein a is 0, 1, 2, 3 or 4; b is 0, 1, 2, 3 or 4; Y² is a direct bond, —O—, —C(O)—, —N(R¹⁶)—, —N(R¹⁶)C(O)— or —C(O)N(R¹⁶)— (wherein R¹⁶ is hydrogen, C₁₋₃alkyl, hydroxyC₂₋₃alkyl, aminoC₂₋₃alkyl or C₁₋₃alkoxyC₂₋₃alkyl) and wherein 1 or 2 of the (CH₂)ₐ or (CH₂)_{b} groups are optionally substituted by 1 or 2 substituents selected from hydroxy and amino and R¹⁵ is as hereinabove defined; provided that when a is 0, then Y² is a single direct bond);
4) N,N-di(C₁₋₄alkyl)carbamoylC₁₋₄alkyl-
(wherein the alkyl groups are independently optionally substituted by 1 or 2 substituents selected from: amino, N—C₁₋₄alkylamino, N,N-di(C₁₋₄alkyl)amino, hydroxy, hydroxyC₁₋₄alkyl, C₁₋₄alkoxy, C₁₋₄alkanoyl, carboxy, sulpho and phosphoryloxy);
provided that:
a) when A is C₁₋₄alkylene and X¹ is of the formula —CO—, —N(R¹⁰)—, —N(R¹⁰)CO— or —CON(R¹⁰)— then when B is R¹², R¹² is defined hereinabove for R¹⁵;
b) when A is C₁₋₄alkylene and X¹ is of the formula —N(R¹⁰)CO—, —CON(R¹⁰)—, or —C(O)O—, then B is not carboxy;
c) when A is C₁₋₄alkylene and X¹ is —CONH— or —NHCO—, then B is not carboxy, hydroxy, phosphoryloxy, amino, N—C₁₋₄alkylamino or N,N-di-C₁₋₄alkylamino;
R⁸ is a group —Y³R¹⁷
(wherein Y³ is a direct bond, —C(O)—, —(O)O—, —N(R¹⁸)—, —C(O)N(R¹⁸)—, —SO₂— or —SO₂NR¹⁸— (wherein R¹⁸ is hydrogen, C₁₋₃alkyl, hydroxyC₂₋₃alkyl, aminoC₂₋₃alkyl or C₁₋₃alkoxyC₂₋₃alkyl) and R¹⁷ is selected from one of the following 4 groups:
1) hydrogen, C₁₋₄alkyl, phenyl, C₁₋₄alkylY⁴C₁₋₄alkyl (wherein Y⁴ is —C(O)—, —NR¹⁹C(O)— or —C(O)NR¹⁹— (wherein R¹⁹ is hydrogen, C₁₋₃alkyl, hydroxyC₂₋₃alkyl, aminoC₂₋₃alkyl or C₁₋₃alkoxyC₂₋₃alkyl));
[which alkyl, alkylY⁴alkyl or phenyl group is optionally substituted by 1 or 2 substituents selected from: halogeno, amino, N—C₁₋₄alkylamino, N,N-di(C₁₋₄alkyl)amino, hydroxy, carboxy, —CON(R²³)R²⁴ (wherein R²³ and R²⁴ are independently selected from hydrogen, C₁₋₃alkyl, hydroxyC₂₋₃alkyl, aminoC₂₋₃alkyl and C₁₋₃alkoxyC₂₋₃alkyl), C₁₋₄alkoxy, C₁₋₄alkoxycarbonylamino, C₁₋₄alkanoyl, sulpho, phosphoryloxy, R¹² (wherein R¹² is as hereinabove defined), and a group —Y⁵R²⁰ [wherein Y⁵ is —NR²¹C(O)— or —OC(O)— (wherein R²¹ represents hydrogen, C₁₋₃alkyl or C₁₋₃alkoxyC₂₋₃alkyl) and R²⁰ is C₁₋₄alkyl or a group R²² (wherein R²² is a 5 or 6 membered aromatic heterocyclic group containing 1 to 4, inclusive, ring heteroatoms selected independently from O, N and S, which aromatic heterocyclic group is optionally substituted by 1 or 2 substituents selected from hydroxy, amino, C₁₋₄alkyl, aminoC₁₋₄alkyl, N—C₁₋₄alkylaminoC₁₋₄alkyl, N,N-di(C₁₋₄alkyl)aminoC₁₋₄alkyl, carboxy, —CONR²⁵R²⁶ and —NR²⁵COR²⁷ (wherein R²⁵ and R²⁶, which may be the same or different, are hydrogen, C₁₋₃alkyl, hydroxyC₂₋₃alkyl, aminoC₂₋₃alkyl or C₁₋₃alkoxyC₂₋₃alkyl and R²⁷ is C₁₋₃alkyl, hydroxyC₂₋₃alkyl, aminoC₂₋₃alkyl or C₁₋₃alkoxyC₂₋₃alkyl))]];
2) R²² (wherein R²² is as hereinabove defined);
3) R²²—C₁₋₄alkyl- (wherein R²² is as here defined); or
4) R¹²Y⁷C₁₋₄alkyl- (wherein R¹² is as hereinabove defined and Y⁷ is —C(O)—, —NR²³C(O)—, —NR²³C(O)C₁₋₄alkyl-, —C(O)NR²³— or —C(O)NR²³C₁₋₄alkyl- (wherein R²³ is as hereinabove defined)) );
and R⁹ is hydrogen or C₁₋₃alkyl;
or a pharmaceutically-acceptable salt, solvate or pro-drug thereof.

2. A compound according to claim 1
where R¹, R² and R³ are all methoxy
or a pharmaceutically-acceptable salt, solvate or pro-drug thereof.

3. A compound according to claim 1 wherein:
R¹, R², and R³ are all C¹⁻⁴alkoxy;
R⁴ and R⁶ are independently selected from hydrogen, hydroxy, C₁₋₃ alkoxy, and C₁₋₃alkyl;
R⁵ is selected from one of the following groups:
1) of the formula -A-X¹—Y¹—B, wherein:
A is ethylene or phenylene;
Y¹ is C₁₋₃alkylene;
X¹ is —CO—, —CON(R¹⁰)—, —N(R¹⁰)—, —N(R¹⁰)CO— or —OC(O)N(R¹⁰)—;
B is carboxy sulpho, phosphoryloxy or of the formula —R¹² (wherein R¹² is piperazinyl, morpholinyl or piperidinyl, each of which is linked via a ring carbon or nitrogen ring atom and each ring is optionally substituted by 1 or 2 substituents selected from C₁₋₄alkyl, C₂₋₄alkanoyl, carbamoyl, cyanoC₁₋₃alkyl, hydroxyC₁₋₃alkyl, carboxyC₁₋₃alkyl and aminoC₁₋₃alkyl);
2) of the formula 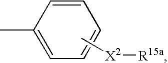
wherein:
the —X²—R^{15a} substituent is in the 3, or 4-position of the phenyl ring;
X² is —(CH₂)ᵣ—;
r is 0, 1 and 2; and
R^{15a} is morpholinyl, piperazinyl, piperidinyl or pyrrolidinyl optionally substituted by 1 or 2 substituents selected from C₁₋₄alkyl, C₂₋₄alkanoyl, carbamoyl, cyanoC₁₋₃alkyl, hydroxyC₁₋₃alkyl, carboxyC₁₋₃alkyl and aminoC₁₋₃alkyl; and substituted by at least 1 substituent selected from C₂₋₄alkanoyl, carbamoyl, N—C₁₋₄ alkylcarbamoyl and N,N-di(C₁₋₄alkyl)carbamoyl;
3) of the formula —(CH₂)ₐ—Y²—(CH₂)_{b}—R^{15b}, wherein:
a is 2 or 3;
b is 0, 1, or 2; and
Y² is a single direct bond, —C(O)—, —NHC(O)— or —C(O)NH—; and
R^{15b} is morpholinyl, piperazinyl, piperidinyl or pyrrolidinyl optionally substituted by 1 or 2 substituents selected from C₁₋₄alkyl, C₂₋₄alkanoyl, carbamoyl, cyanoC₁₋₃alkyl, hydroxyC₁₋₃alkyl, carboxyC₁₋₃alkyl and aminoC₁₋₃alkyl; and substituted by at least 1 substituent selected from C₂₋₄alkanoyl, carbamoyl, N—C₁₋₄ alkylcarbamoyl and N,N-di(C₁₋₄alkyl)carbamoyl;
or
4) N,N-di(C₁₋₄alkyl)carbamoylC₁₋₄alkyl-, wherein the alkyl group is optionally substituted by 1 or 2 substituents selected from amino, N-methylamino, N—N-dimethylamino, hydroxy, methoxy, carboxy, sulpho and phosphoryloxy; and
R⁸ is a group —Y³R¹⁷ (wherein Y³ is —C(O)—, —C(O)O— or —C(O)NH—; and
R¹⁷ is selected from one of the following 4 groups:
1) hydrogen, C₁₋₄alkyl, phenyl or C₁₋₄alkylY⁴C₁₋₄alkyl (wherein Y⁴ is —NHCO— or —CONH—); [which alkyl, alkylY⁴alkyl or phenyl group is optionally substituted by 1 or 2 substituents selected from: halogeno, amino, N—C₁₋₄alkylamino, N,N-di(C₁₋₄alkyl)amino, C₁₋₄alkoxy, C₁₋₄alkoxycarbonylamino, C₁₋₄alkanoyl, phosphoryloxy, R^{12a} (wherein R^{12a} is piperazinyl, morpholinyl or piperidinyl, each of which is linked via a ring carbon or nitrogen ring atom and each ring is optionally substituted by 1 or 2 of the substituents selected from C¹⁻⁴alkyl, C₂₋₄alkanoyl, carbamoyl, cyanoC₁₋₃alkyl, hydroxyC₁₋₃alkyl, carboxyC₁₋₃alkyl and aminoC₁₋₃alkyl), —Y⁵—R²⁰ [wherein Y⁵ is —NHCO—; and R²⁰is C₁₋₄alkyl or R^{22a} (wherein R^{22a}is imidazolyl, pyridyl, pyrimidyl, thiazolyl or pyrazinyl, each of which is optionally substituted by C₁₋₄alkyl)]];
2) R^{22a} (wherein R^{22a} is as hereinabove defined);
3) R^{22a}—C₁₋₄alkyl- (wherein R^{22a} is as hereinabove defined); or
4) R^{12a}Y⁷C₁₋₄alkyl- (wherein R^{12a} is as hereinabove defined and Y⁷ is Y⁷ is —NHC(O)— or —CONH—));
and R⁹ is hydrogen;
or a pharmaceutically-acceptable salt, solvate or pro-drug thereof.

4. A compound according to claim 1 wherein:
R¹, R², and R³ are all methoxy;
R⁴ and R⁶ are independently selected from hydrogen, hydroxy, methoxy and methyl;
R⁵ is selected from one of the following groups:
1) of the formula -A-X¹—Y¹—B, wherein:
A is ethylene or phenylene;
Y¹ is C₁₋₃alkylene;
X¹ is —CO—, —CON(R¹⁰)—, —N(R¹⁰)—, —N(R¹⁰)CO— or —OC(O)N(R¹⁰)—, wherein R¹⁰ is as defined in claim 1;
B is carboxy sulpho, phosphoryloxy or of the formula —R¹² (wherein R¹² is piperazinyl, morpholinyl or piperidinyl, each of which is linked via a ring carbon or nitrogen ring atom and each ring is optionally substituted by 1 or 2 substituents selected from C₁₋₄ alkyl, C₂₋₄alkanoyl, carbamoyl, cyanoC₁₋₃alkyl, hydroxyC₁₋₃alkyl, carboxyC₁₋₃alkyl and aminoC₁₋₃alkyl);
2) of the formula 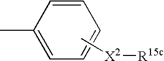
wherein:
the —X²—R^{15c} substituent is in the 3, or 4-position of the phenyl ring;
X² is —(CH₂)ᵣ—;
r is 0, and 2; and
R^{15c} is morpholinyl, piperazinyl or piperidinyl optionally substituted by 1 or 2 substituents selected from C₁₋₄ alkyl, C₂₋₄alkanoyl, carbamoyl, cyanoC₁₋₃alkyl, hydroxyC₁₋₃alkyl, carboxyC₁₋₃alkyl and aminoC₁₋₃alkyl; and substituted by at least 1 substituent selected from C₂₋₄alkanoyl, carbamoyl, N—C₁₋₄alkylcarbamoyl and N,N-di(C₂₋₄alkyl)carbamoyl;
3) of the formula —(CH₂)ₐ—Y²—(CH₂)_{b}—R^{15d}, wherein:
a is 2 or 3;
b is 0 or 1; and
Y² is a single direct bond, —C(O)— or —NHC(O)—; and
R^{15d} is morpholinyl, piperazinyl or piperidinyl optionally substituted by 1 or 2 substituents selected from C₁₋₄ alkyl, C₂₋₄alkanoyl, carbamoyl, cyanoC₁₋₃alkyl, hydroxyC₁₋₃alkyl, carboxyC₁₋₃alkyl and aminoC₁₋₃alkyl; and substituted by at least 1 substituent selected from C₂₋₄alkanoyl, carbamoyl, N—C₁₋₄alkylcarbamoyl and N,N-di(C₁₋₄alkyl)carbamoyl; or
4) N,N-di(C₁₋₄alkyl)carbamoylC₁₋₄alkyl-, wherein the alkyl group is optionally substituted by 1 or 2 substituents selected from amino, N-methylamino, N—N-dimethylamino, hydroxy, methoxy, carboxy, sulpho and phosphoryloxy; and
R⁸ is a group —Y³R¹⁷ (wherein Y³ is —C(O)— or —C(O)O—; and
R¹⁷ is selected from one of the following 4 groups:
1) C₁₋₄alkyl [which alkyl, group is optionally substituted by 1 or 2 substituents selected from: fluoro, chloro and bromo];
2) R^{22b} (wherein R^{22b} is imidazolyl, pyridyl, pyrimidyl, thiazolyl or pyrazinyl, each of which is optionally substituted by C₁₋₄alkyl);
3) R^{22b} —C₁₋₄alkyl- (wherein R^{22b} is as hereinabove defined); or
4) R^{12b}Y⁷C₁₋₄alkyl- (wherein R^{12a} is morpholinyl, piperidinyl or piperazinyl each of which is optionally substituted by methyl, ethyl, acetyl, propionyl, carbamoyl or 2-hydroxyethyl; and Y⁷ is —NHC(O)— or —CONH—));
and R⁹ is hydrogen;
or a pharmaceutically-acceptable salt, solvate or pro-drug thereof.

5. A compound of formula (II): 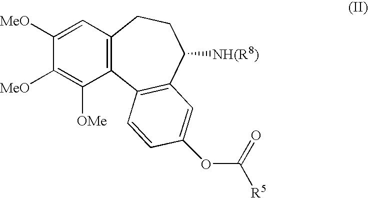
wherein R⁵ and R⁸ are as defined in claim 1;
or a pharmacetically-acceptable salt, solvate or pro-drug thereof.

6. A compound of the formula (III) 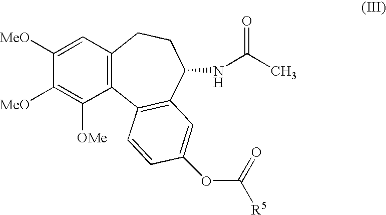
wherein:
R⁵ is selected from one of the following,groups:
1) of the formula -A-X¹—Y¹—B, wherein:
A is ethylene or phenylene;
Y¹ is C₁₋₃alkylene;
X¹ is —CO—, —CONH—, —NH—, —NHCO— or —OC(O)NH—;
B is carboxy sulpho, phosphoryloxy or of the formula —R¹² (wherein R¹² is piperazinyl, morpholinyl or piperidinyl, each of which is linked via a ring carbon or nitrogen ring atom and each ring is optionally substituted by 1 or 2 of the substituents selected from C₁₋₄ alkyl, C₂₋₄alkanoyl, carbamoyl, cyanoC₁₋₃alkyl, hydroxyC₁₋₃alkyl, carboxyC₁₋₃alkyl and aminoC₁₋₃alkyl);
2) of the formula 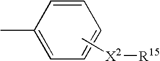
wherein:
the —X²—R¹⁵ substituent is in the 3, or 4-position of the phenyl ring;
X² is —(CH₂)ᵣ—;
r is, 0, 1 and 2; and
R¹⁵ is morpholinyl, piperazinyl, piperidinyl or pyrrolidinyl optionally substituted as immediately hereinabove defined for R¹², and substituted by at least 1 substituent selected from C₂₋₄alkanol, carbamoyl, N—C₁₋₄alkylcarbamoyl and N,N-di(C₁₋₄alkyl)carbamoyl;
3) of the formula —(CH₂)ₐ—Y²—(CH₂)_{b}—R¹⁵, wherein:
a is 2 or 3;
b is 0, 1, or 2; and
Y² is a single direct bond, —C(O)—, —NHC(O)— or —C(O)NH—; or
4) N,N-di(C₁₋₄alkyl)carbamoylC₁₋₄alkyl-, wherein the alkyl group is optionally substituted by 1 or 2 substituents selected from amino, N-methylamino, N—N-dimethylamino, hydroxy, methoxy, carboxy, sulpho and phosphoryloxy;
or a pharmaceutically acceptable salt, solvate or prodrug thereof.

7. A compound according to claim 6 wherein:
R⁵ is selected from one of the following groups:
1) of the formula -A-X¹—Y¹—B, wherein:
A is ethylene or phenylene;
Y¹ is C₁₋₃alkylene;
X¹ is —CO—, —NHCO—;
B is carboxy sulpho, phosphoryloxy or of the formula —R¹² (wherein R¹² is
piperazinyl, morpholinyl or piperidinyl, each of which is linked via a ring carbon or nitrogen ring atom and each ring is optionally substituted by 1 or 2 of the substituents selected from C₁₋₄ alkyl, C₂₋₄alkanoyl, carbamoyl, cyanoC₁₋₃alkyl, hydroxyC₁₋₃alkyl, carboxyC₁₋₃alkyl and aminoC₁₋₃alkyl);
2) of the formula 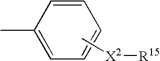
wherein:
the —X²—R¹⁵ substituent is in the 3, or 4-position of the phenyl ring;
X² is —(CH₂)ᵣ—;
r is 1 and 2; and
R^{15a} is morpholinyl, piperazinyl, piperidinyl or pyrrolidinyl optionally substituted by 1 or 2 substituents selected from C₁₋₄alkyl, C₂₋₄alkanoyl, carbamoyl, cyanoC₁₋₃alkyl, hydroxyC₁₋₃alkyl, carboxyC₁₋₃alkyl and aminoC₁₋₃alkyl; and substituted by at least 1 substituent selected from C₂₋₄alkanoyl, carbamoyl, N—C₁₋₄ alkylcarbamoyl and N,N-di(C₁₋₄alkyl)carbamoyl;
3) of the formula —(CH₂)ₐ—Y²—(CH₂)_{b}—R^{15b}, wherein:
a is 2 or 3;
b is 0; and
Y² is a single direct bond, —C(O)—, —NHC(O)— or —C(O)NH—; and
R^{15b} is morpholinyl, piperazinyl, piperidinyl or pyrrolidinyl optionally substituted by 1 or 2 substituents selected from C₁₋₄alkyl, C₂₋₄alkanoyl, carbamoyl, cyanoC₁₋₃alkyl, hydroxyC₁₋₃alkyl, carboxyC₁₋₃alkyl and aminoC₁₋₃alkyl; and substituted by at least 1 substituent selected from C₂₋₄alkanoyl, carbamoyl, N—C₁₋₄ alkylcarbamoyl and N,N-di(C₁₋₄alkyl)carbamoyl;
or
4) N,N-di(C₁₋₄alkyl)carbamoylC₁₋₄alkyl-, wherein the alkyl group is optionally substituted by 1 or 2 substituents selected from amino, hydroxy and phosphoryloxy;
or a pharmaceutically acceptable salt, solvate or pro-drug thereof.

8. A compound according to claim 6 wherein:
R⁵ is selected from one of the following groups:
1) of the formula -A-X¹—Y¹—B, wherein:
A is phenylene;
X¹ is —CO—, —NHCO—;
Y¹ is methylene or ethylene;
B is piperazino or morpholinyl each of which is linked via a ring nitrogen atom and each ring is optionally substituted by 1 methyl or acetyl group;
2) of the formula 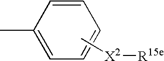
wherein:
the —X²—R^{15e} substituent is in the 3, or 4-position of the phenyl ring;
X² is —(CH₂)ᵣ—;
r is 1; and
R^{15e} is piperazino or morpholinyl each of which is linked via a ring nitrogen atom and each ring is optionally substituted by 1 methyl or acetyl group;
3) of the formula —(CH₂)ₐ—Y²—(CH₂)_{b}—R^{15f}, wherein:
a is 2 or 3;
b is 0; and
Y²is —C(O)—;
R^{15f} is piperazino or morpholinyl each of which is linked via a ring nitrogen atom and each ring is optionally substituted by 1 methyl or acetyl group; or
4) 2-[N,N-di(C₁₋₄alkyl)carbamoyl]ethyl- or 3-[N,N-di(C₁₋₄alkyl)carbamoyl]propyl-, wherein the C₁₋₄alkyl group is optionally substituted by 1 hydroxy group;
or a pharmaceutically acceptable salt, solvate or prodrug thereof.

9. A compound according to claim 6 wherein:
R⁵ is 2-[N,N-di(C₁₋₄alkyl)carbamoyl]ethyl-, or 3-[N,N-di(C₁₋₄alkyl)carbamoyl]propyl-, wherein the C₁₋₄alkyl group is optionally substituted by 1 hydroxy group;
or a pharmaceutically acceptable salt, solvate or prodrug thereof.

10. A compound selected from:
(5S)-5-acetylamino-9,10,11-trimethoxy-6,7-dihydro-5H-dibenzo[a,c]cyclohepten-3-yl 5-(4-acetylpiperazin-1-yl)-5-oxopentanoate;
(5S)-5-acetylamino-9,10,11-trimethoxy-6,7-dihydro-5H-dibenzo[a,c}cyclohepten-3-yl 4-(4-acetylpiperazin-1-yl)-4-oxobutanoate;
(5S)-5-acetylamino-9,10,11-trimethoxy-6,7-dihydro-5H-dibenzo[a,c]cyclohepten-3-yl 3-(4-acetylpiperazin-1-ylmethyl)benzoate;
(5S)-5-acetylamino-9,10,11-trimethoxy-6,7-dihydro-5H-dibenzo[a,c]cyclohepten-3-yl 4-[3-(4-methylpiperazin-1-yl)propionylamino]benzoate;
(5S)-5-acetylamino-9,10,11-trimethoxy-6,7-dihydro-5H-dibenzo[a,c]cyclohepten-3-yl 3-(4-carbamoylpiperazin-1-ylmethyl)benzoate;
(5S)-5-acetylamino-9,10,11-trimethoxy-6,7-dihydro-5H-dibenzo[a,c]cyclohepten-3-yl N-acetylpiperidin-1-ylcarboxylate;
(5S)-5-acetylamino-9,10,11-trimethoxy-6,7-dihydro-5H-dibenzo[a,c]cyclohepten-3-yl 3-[N,N-di-(2-hydroxyethyl)carbamoyl]propanoate; and
(5S)-5-acetylamino-9,10,11-trimethoxy-6,7-dihydro-5H-dibenzo[a,c]cyclohepten-3-yl 4-[N,N-di(2-hydroxyethyl)carbamoyl]butanoate;
and pharmaceutically-acceptable salts, solvates and pro-drugs thereof.

11. A pharmaceutical composition comprising a compound according to any one of claims 1 to 10 or pharmaceutically acceptable salt, solvate or pro-drug thereof, in association with a pharmaceutically acceptable carrier.

12-13. (canceled)

14. A process for preparing a compound of the formula (I), or a compound of the formula (I) wherein at least 1 functional group is protected, wherein R¹, R², R³ R⁴, R⁵, R⁶, R⁷, R⁸, R⁹, R¹⁰, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, A, B, Q, X¹, X², Y¹, Y², Y³, Y⁴, Y⁵, Y⁷, p and r are as defined in claim 1, comprising:
(a) reacting a compound of the formula (X): 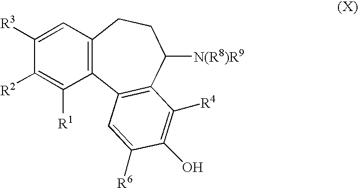
with a compound of the formula R⁵—COOH or an activated derivative thereof;
(b) when R⁵ is of the formula: 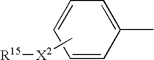
reacting a compound of the formula (XI): 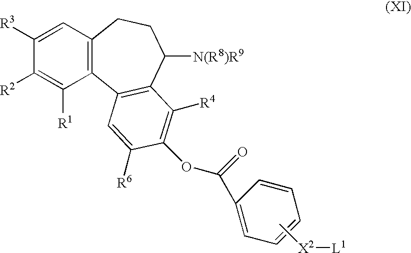
with R¹⁵ (wherein L¹ is a leaving group)
(c) introducing substituents onto a ring nitrogen atom in R¹² or R¹⁵;
(d) converting one compound of the formula (I) into another compound of the formula (I);
(e) optionally forming a phosphoryloxy group by reacting the corresponding hydroxy compound with a phosphoramidite;
wherein any functional groups are optionally protected.
and thereafter optionally:
i) converting a compound of formula (I) into another compound of formula (I);
ii) removing any protecting groups;
iii) forming a pharmaceutically acceptable salt, solvate or pro-drug thereof.

15. A method for reducing neovascularization by selectively damaging newly formed vascular endothelium in a warm-blooded animal in need thereof, which comprises administering to said animal an effective amount of a compound according to any one of claims 1 to 10 or a pharmaceutically-acceptable salt, solvate or pro-drug thereof.

16. A method for reducing neovascularization by selectively damaging newly formed vascular endothelium in a warm-blooded animal in need thereof, which comprises administering to said animal, in divided doses, a compound according to any one of claims 1 to 10 or a pharmaceutically-acceptable salt, solvate or pro-drug thereof.
